(19) United States
(12) Patent Application Publication
(10) Pub. No.: US 2025/0322463 A1
(43) Pub. Date: Oct. 16, 2025

(54) SYSTEMS AND METHODS FOR GENERATING FINANCIAL INDEXES FOR MEDICAL CONDITIONS

(71) Applicant: IMX, Inc., Las Vegas, NV (US)

(72) Inventors: Juan Diego Gomez, Las Vegas, NV (US); James Plante, Las Vegas, NV (US)

(21) Appl. No.: 18/865,200

(22) PCT Filed: Feb. 13, 2023
(86) PCT No.: PCT/US2023/062496

§ 371 Date: Nov. 12, 2024

Related U.S. Application Data
(60) Provisional application No. 63/341,847, filed on May 13, 2022.
(60) Provisional application No. 63/418,387, filed on Oct. 21, 2022.
(60) Provisional application No. 63/422,924, filed on Nov. 4, 2022.

(51) Int. Cl. G06Q 40/08; G16H 15/00; G16H 70/40

(52) U.S. Cl. CPC G06Q 40/08; G16H 15/00; G16H 70/40

(57) ABSTRACT

Techniques for generating an index that is statistically sensitive to a cost of treating a medical condition are disclosed. Claim forms that are related to an identified medical condition are accessed. Codes are identified from within the claim forms. These codes are determined to be procedure codes. Pharmaceuticals for the procedure codes are identified, and a cost for those pharmaceuticals is determined. Each of the procedure codes is weighted based on whether each procedure code is directly related to the particular medical condition or is related to an identified co-morbidity of the particular medical condition. A cost for the weighted procedure codes is determined. The cost for the weighted procedure codes and the cost for the pharmaceuticals are used to determine a per capita cost for the medical condition. An index for the medical condition is generated based on the per capita cost.

### INCORPORATION BY SPECIFIC REFERENCE

This application claims the benefit of and priority to U.S. Provisional Patent Application Ser. No. 63/341,847 filed on May 13, 2022 and entitled “SYSTEMS AND METHODS FOR GENERATING FINANCIAL INDEXES FOR MEDICAL CONDITIONS,” which application is expressly incorporated herein by reference in its entirety. This application claims the benefit of and priority to U.S. Provisional Patent Application Ser. No. 63/418,387 filed on Oct. 21, 2022 and entitled “USING NATURAL LANGUAGE PROCESSING TO GENERATE A DRUG AND MEDICAL CONDITION LIBRARY,” which application is expressly incorporated herein by reference in its entirety. This application claims the benefit of and priority to U.S. Provisional Patent Application Ser. No. 63/422,924 filed on Nov. 4, 2022 and entitled “SYSTEMS AND METHODS FOR GENERATING FINANCIAL INDEXES FOR MEDICAL CONDITIONS,” which application is expressly incorporated herein by reference in its entirety.

### BACKGROUND

The price of many commodities can fluctuate significantly within a relatively short period of time. For instance, oil prices rose from $18.50 per barrel in January 1999 to over $147 per barrel in June 2008. A futures contract can help stabilize the market for a particular good.

A futures market (or simply “future”) is essentially an auction-based market that allows participants to buy and sell goods for delivery at a future point in time. That is, a future is an exchange-traded derivative contract that locks in the future delivery of an item for a price that is set today.

Prior to the availability of the futures market, oil was a one-sided market in that the oil producers set the price, and the consumers were forced to pay that prior. Once a futures market was developed for the oil industry, the price of oil generally stabilized.

Healthcare is currently a one-sided market because the payers generally have complete control over the terms. The cost of healthcare has been rising at a significant rate in recent years. Furthermore, the healthcare industry generally recognizes a vulnerability with regard to price shocks from new pharmaceuticals.

As an example, several new treatments for specific diseases have been approved, where these treatments often extend and improve the quality of a patient's life. Those treatments, however, are very expensive. As an example, consider the drug Sovaldi. Sovaldi is designed to treat and even cure hepatitis C. This drug can cost about $40,000 for a full treatment. The cost of treating hepatitis C prior to Sovaldi was about $500,000 for a full treatment.

From the insurance company's perspective, the insurance company would much rather pay $40,000 as compared to $500,000 to treat hepatitis C. Creating a futures market would provide equal footing to providers, treatment manufacturers, and ultimately patients. A futures market would allow manufacturers to sell treatments (e.g., drugs) into a futures market before those treatments were even manufactured. Doing so would greatly reduce the cost of the treatment.

A futures market typically relies on a so-called market “index.” An index is a hypothetical investment portfolio that generally represents the holdings of a portion of a financial market. Participants in the futures market rely on the index when considering whether to buy or sell a futures contract because the index helps evaluate the cost of a good.

Futures contracts are available for many goods, such as oil, because a robust index has been created for such commodities. For healthcare, however, there is no index and thus there is no futures market in the healthcare industry. A futures market in the healthcare industry could significantly help stabilize the ever increasing costs of healthcare. To achieve the futures market, however, an index is needed for the cost of healthcare.

To be complete, the index should account not only for the services that are expended when treating a disease but also for the drugs or pharmaceuticals that are prescribed and used to treat a disease. There are various techniques for linking a pharmaceutical to a medical condition.

One technique (i.e. a naïve technique) involves identifying all of the patients who have been diagnosed with a medical condition and then subsequently identifying all of the pharmaceuticals that those patients are taking. Those pharmaceuticals could then be ranked based on frequency or perhaps popularity of use. If a drug has a sufficiently high popularity, then that drug can be linked with the medical condition as either a primary drug for that medical condition or a drug used to treat a co-morbidity or associated disease that is often linked with the primary medical condition. Such an approach is wrought with false positives and false negatives. This approach also introduces a significant amount of noise into the analysis because not all drugs that a person who has a medical condition is taking may be geared specifically for that medical condition. For instance, a diabetic person might take medicine to treat diabetes, but that person might also take other medicine to treat other ailments.

Another technique involves a statistical-based analysis. This technique generally involves analyzing each drug and determining a likelihood of seeing a particular drug among patients who have a particular medical condition. The analysis also includes evaluating the likelihood of seeing that drug being used by patients who have not been diagnosed with the medical condition. If the drug is rarely (if ever) used by patients that do not have the medical condition, then there is a reasonably high likelihood that the drug is used to treat the medical condition. On the other hand, if the drug is frequently used even by patients who do not have the medical condition, then there is less certainty as to whether that drug used to treat the medical condition.

As shown above, there are various techniques for linking a pharmaceutical to a medical condition. Such techniques, however, have numerous weaknesses and thus are not optimal. What is needed, therefore, is an improved technique for linking pharmaceuticals to medical conditions. Doing so will greatly benefit the generation of a robust index that can be used in the healthcare industry for determining the costs, or at least the cost fluctuations, associated with a medical condition.

The subject matter claimed herein is not limited to embodiments that solve any disadvantages or that operate only in environments such as those described above. Rather, this background is only provided to illustrate one exemplary technology area where some embodiments described herein may be practiced.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates an example of a professional claim form.

FIG. 2 illustrates how the professional claim form includes both diagnostic codes and procedure codes.

FIG. 3 illustrates an example of an institutional claim form.

FIG. 4 illustrates how the institutional claim form includes procedure codes but does not include diagnostic codes.

FIGS. 5A and 5B illustrate flowcharts of an example method for generating an index that is statistically sensitive to a cost of treating a medical condition.

FIG. 6 illustrates an architecture that can be used to generate the index.

FIG. 7 illustrates different sets of claim forms and a weekly pattern that is detected within those forms.

FIG. 8 illustrates a monthly pattern that is detected within the forms.

FIG. 9 illustrates how some of the submitted claim forms are professional claim forms.

FIG. 10 illustrates an impact factor that is used to weight the procedure codes.

FIG. 11 illustrates a smoothed function that is achieved based on the impact factor.

FIG. 12 illustrates a mapping operation.

FIG. 13 illustrates a per capita analysis chart.

FIG. 14 illustrates costs associated with pharmaceuticals.

FIG. 15 illustrates a combined analysis chart.

FIG. 16 illustrates a normalized index.

FIG. 17 illustrates the normalized index based on different time factors.

FIG. 18 illustrates indexes for other medical conditions.

FIG. 19 illustrates an example architecture designed to obtain data about different pharmaceuticals (aka drugs) for a medical condition.

FIG. 20 illustrates how a drug and medical condition library can be generated.

FIG. 21 illustrates an example user interface that may be displayed and that can be used to display information for pharmaceuticals for a medical condition.

FIGS. 22A and 22B illustrate a flowchart of an example method for generating a drug and medical condition library.

FIG. 23 illustrates an example computer system that can be configured to perform any of the disclosed operations.

### DETAILED DESCRIPTION

Embodiments disclosed herein relate to systems, devices, and methods for generating an index that is statistically sensitive to a cost of treating a medical condition. The index is sensitive to not only the cost of service for treating a condition but also the cost for pharmaceuticals that are used to treat the condition.

Some embodiments access a set of claim forms that are related to an identified medical condition. The embodiments identify, from within the set of claim forms, codes that are determined to be procedure codes. A determination is made as to whether one or more pharmaceuticals are associated with each procedure code in the procedure codes. For procedure codes that have associated pharmaceuticals, the embodiments determine a cost for the pharmaceuticals. The embodiments also weight each of the procedure codes based on whether each procedure code is a descriptor for the particular medical condition or is a descriptor for an identified co-morbidity of the particular medical condition. The embodiments determine a cost for the weighted procedure codes. The embodiments use the cost for the weighted procedure codes and the cost for the pharmaceuticals to determine a per capita cost for the medical condition. The embodiments then generate an index for the medical condition based on the per capita cost.

Some embodiments access a plurality of claim forms for patients who are identified as having a particular medical condition. The claim forms include claim forms related to the particular medical condition and claim forms that are not related to the particular medical condition. Some embodiments filter the claim forms to remove claim forms that are not related to the particular medical condition. The filtering is achieved by performing image or object segmentation to identify a plurality of claim codes from among the claim forms. The filtering further includes, for each identified claim code, performing a syntactical analysis on a corresponding description for each identified claim code to determine whether each identified claim code is related to the particular medical condition.

Based on the syntactical analysis, the filtering further includes removing claim forms that do not have at least one claim code related to the particular medical condition while preserving claim forms that do have at least one claim code related to the particular medical condition even if claim forms that do have at least one claim code related to the particular medical condition have other claim codes that are not related to the particular medical condition. As a result, a set of claim forms remain, and the set of claim forms include claim codes that are related to the particular medical condition and claim codes that are suspected of being co-morbidities to the particular medical condition.

Some embodiments also identify, from within the set of claim forms, codes that are determined to be procedure codes. Some embodiments weight each procedure code based on whether the procedure code is related to the particular medical condition or is related to a co-morbidity of the particular medical condition. This weighting is performed by applying an impact factor (or contribution factor) to the procedure codes. In some embodiments, the impact factor (or contribution factor) is defined as a number of times that the procedure code is identified as being associated with a diagnostic code for the particular medical condition within the set of claim forms divided by a total number of times that the procedure code is identified within the set of claim forms. Some embodiments determine a cost for each weighted procedure code. Some embodiments use the cost for each weighted procedure code to determine a per capita cost and then generate an index based on the per capita cost.

In some embodiments, the impact factor (or contribution factor) is calculated on “professional claims,” which include both procedural codes (procedural code data) and corresponding diagnostic codes (diagnostic code data), and is then extrapolated to “institutional” claims, which include only procedural codes (procedural code data), where the calculation of an impact factor would be impossible (due to the lack of linkage diagnosis/procedure). By way of illustration, and without being bound to any particular theory, in some embodiments, due to the nature of both pluralities of claims (professional and institutional), an impact factor can be calculated deterministically only on the first plurality. Therefore, the impact factor weighing the second plurality is a heuristic inference extrapolated from the first to the second plurality. In machine learning (or artificial intelligence; AI), this procedure may be referred to as “transfer learning” or “knowledge transfer”.

Some embodiments can include method (e.g., a method for generating an index that is statistically sensitive to a cost of treating a medical condition).

In some embodiments, the method can comprise accessing a first plurality of patient medical claims.

In some embodiments, each of the first plurality of patient medical claims comprises one or more procedure code(s) and a corresponding one or more diagnostic code(s) associated with a respective one or more procedure code(s).

In some embodiments, each of the one or more procedure code(s) relates to a respective medical procedure, service, or supply and is associated with at least one of: a diagnostic code that is related to a particular medical condition; and a diagnostic code that is not related to the particular medical condition.

In some embodiments, the method can comprise filtering the first plurality of patient medical claims to (i) remove claims that do not include at least one of the one or more procedure code(s) that is associated with a diagnostic code that is related to the particular medical condition and/or (ii) retain (only) claims that include at least one of the one or more procedure code(s) that is associated with a diagnostic code that is related to the particular medical condition, thereby generating a first subset of patient medical claims that are related to the particular medical condition.

In some embodiments, the method can comprise weighting each of the one or more procedure code(s) of the first subset of patient medical claims to determine whether each of the one or more procedure code(s) of the first subset of patient medical claims is related to the particular medical condition or is related to a co-morbidity of the particular medical condition.

In some embodiments, said weighting is performed by applying an impact factor to each of the one or more procedure code(s) of the first subset of patient medical claims.

In some embodiments, the impact factor is defined as a number of times that said each of the one or more procedure code(s) of the first subset of patient medical claims is identified as being associated with at least one diagnostic code that is related to the particular medical condition divided by a total number of times that said each of the one or more procedure code(s) is identified within the first subset of patient medical claims, thereby producing a first set of weighted procedure codes.

In some embodiments, the method can comprise accessing a second plurality of patient medical claims.

In some embodiments, each of the second plurality of patient medical claims comprises one or more undesignated procedure code(s) that do not have a corresponding diagnostic code.

In some embodiments, the method can comprise optionally filtering the second plurality of patient medical claims to (i) remove claims that do not include at least one of the one or more procedure code(s) from the first subset of patient medical claims and/or (ii) retain (only) claims that include at least one of the one or more procedure code(s) from the first subset of patient medical claims, thereby generating a second subset of patient medical claims that are related to the particular medical condition.

In some embodiments, the method can comprise weighting each of the one or more procedure code(s) of the second subset of patient medical claims to determine whether each of the one or more procedure code(s) of the second subset of patient medical claims is related to the particular medical condition or is related to a co-morbidity of the particular medical condition.

In some embodiments, said weighting is performed by applying the impact factor (as determined, above) to each of the one or more procedure code(s) of the second subset of patient medical claims, thereby producing a second set of weighted procedure codes.

In some embodiments, the method can comprise determining a cost for each medical procedure, service, or supply related to each of said one or more procedure code(s) in the first set of weighted procedure codes and the second set of weighted procedure codes based on a cost of said each of the one or more procedure code(s) reflected in the first plurality of patient medical claims and the second plurality of patient medical claims.

In some embodiments, the method can comprise using the cost for each medical procedure, service, or supply to determine a per capita cost for the particular medical condition.

In some embodiments, the method can comprise generating a financial index based on or representing the per capita cost for the particular medical condition.

Some embodiments cause a big data and machine learning (BD/ML) engine to obtain information describing multiple pharmaceuticals. The information includes, for each respective pharmaceutical, at least a name of each pharmaceutical. The embodiments cause the BD/ML engine to obtain a national drug code (NDC) for each pharmaceutical. An NDC is a product identifier that is assigned by manufacturers and packagers of drugs. If a manufacturer packages the same medication in different sizes, each size will receive a different NDC. If multiple manufacturers produce the same drug, then each manufacturer will use a different NDC. In this regard, the NDC is provided to help identify a particular drug.

The embodiments also cause the BD/ML engine to use the NDC for each pharmaceutical to execute a website query in an attempt to identify a textual description associated with each pharmaceutical. A result of executing the website query returns a first set of textual descriptions for a first subset of the pharmaceuticals. Notably, a second subset of pharmaceuticals remains, where the second subset includes pharmaceuticals for queries that did not return textual descriptions. For pharmaceuticals included in the second subset, the embodiments cause the BD/ML engine to use the names of the pharmaceuticals in the second subset as a parameter in a search engine query. A result of executing the search engine query returns a second set of textual descriptions for a third subset of the pharmaceuticals. Notably, a fourth subset of pharmaceuticals remains, where the fourth subset includes pharmaceuticals for search engine queries that did not return textual descriptions. For pharmaceuticals included in the fourth subset, the embodiments cause the BD/ML engine to use the NDCs for each pharmaceutical in the fourth subset as a parameter in a second website query. A result of executing the second website query returns historical data comprising an RxNorm Concept Unique Identifier (RxCUI) for a fifth subset of the pharmaceuticals. The RxNorm creates a standard set of identifiers for the various combinations of strengths, doses, and even ingredients for a drug. All of the drugs that include the same strengths, same active ingredients, and same doses will have the same RxNorm name. The RxCUI is an entirely unique identifier that is provided to a particular drug entry in the RxNorm. The RxCUI links one entity in RxNorm to all other related entities. The RxCUIs are subsequently used as parameters in a third website query, and a result of executing the third website query returns a third set of textual descriptions for the pharmaceuticals in the fifth subset. The embodiments compile the first set of textual descriptions, the second set of textual descriptions, and the third set of textual descriptions into a compiled set of textual descriptions. The embodiments cause the BD/ML engine to parse the compiled set of textual descriptions to identify linkages between pharmaceuticals and medical conditions. The BD/ML engine then generates a drug and medical condition library that links pharmaceuticals to medical conditions based on the identified linkages. Natural language processing and other big data and machine learning can be implemented throughout these processes.

The embodiments are able to use the drug and medical condition library to generate an index that tracks the costs of pharmaceuticals that are used to treat various medical conditions. This information can then be coupled with the service cost information described above. Together, these pieces of information can be used to generate an index that tracks the service and pharmaceutical costs to treat a medical condition.

### Examples of Technical Benefits, Improvements, and Practical Applications

The following section outlines some example improvements and practical applications provided by the disclosed embodiments. It will be appreciated, however, that these are just examples only and that the embodiments are not limited to only these improvements.

The disclosed embodiments are focused on creating a series of indexes that represent the cost of treating individual diseases. Any type of disease can be indexed. Examples of such diseases include, but certainly are not limited to, diabetes, Alzheimer's, cardiovascular disease, hepatitis, cancer, and many others. These disclosed indexes can optionally serve as the basis for capital market products that will be publicly traded. By providing these indexes, the embodiments can help stabilize the markets and mitigate significant surges in price.

The disclosed embodiments beneficially rely on machine learning and big data analysis to generate a healthcare related index, which is something that has not been achievable until now. The embodiments are able to obtain access to large samples of patient data for any type of disease, where that data is anonymized to protect privacy. By “large” samples of patient data, it is typically meant that more than 1 million claims are analyzed. Big data analysis is performed on that large sampling of data. This index can track the service-related costs.

The disclosed embodiments also beneficially rely on machine learning, natural language processing, and big data analysis to generate the drug and medical condition library, which is something that has not been achievable until now. The embodiments are able to obtain access to large samples of patient data for any type of disease, where that data is anonymized to protect privacy. By “large” samples of patient data, it is typically meant that more than 1 million claims are analyzed. Big data analysis is performed on that large sampling of data. The data can then be analyzed to identify pharmaceuticals and how those pharmaceuticals relate to specific medical conditions. This index can track the pharmaceutical costs.

The embodiments utilize unique algorithms to represent the cost of any type of treatment to thereby generate a market index for a medical condition, where the index is based on various linkages that are identified between medicine/drugs and medical conditions as well as service expenses. Optionally, the disclosed indexes can be used to underlie futures contracts, thereby leading to market stability. Industry participants, physicians, and even patients can use the disclosed indexes to manage the costs for healthcare. The embodiments beneficially create a series of indexes that are designed to capture the annualized cost of various healthcare treatments. Accordingly, these and numerous other benefits will now be described in more detail throughout the remaining portions of this disclosure.

### Example Claim Forms

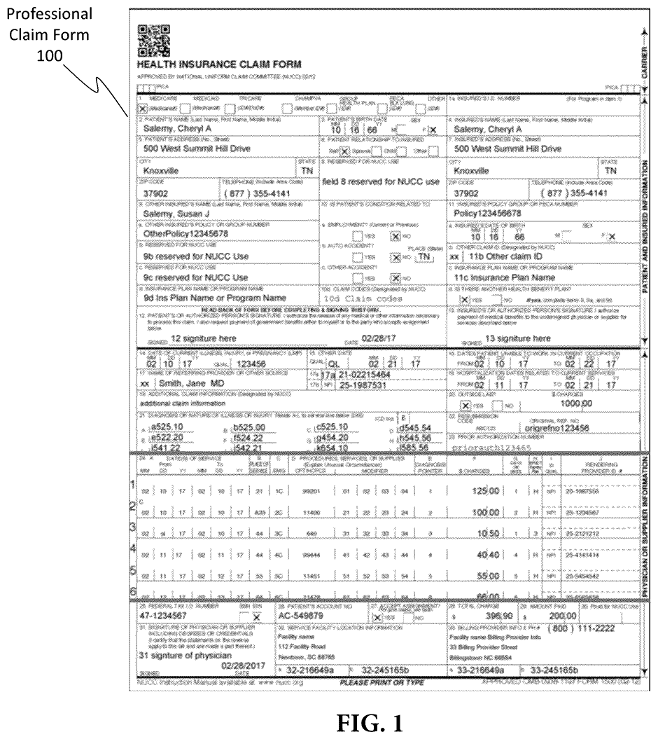

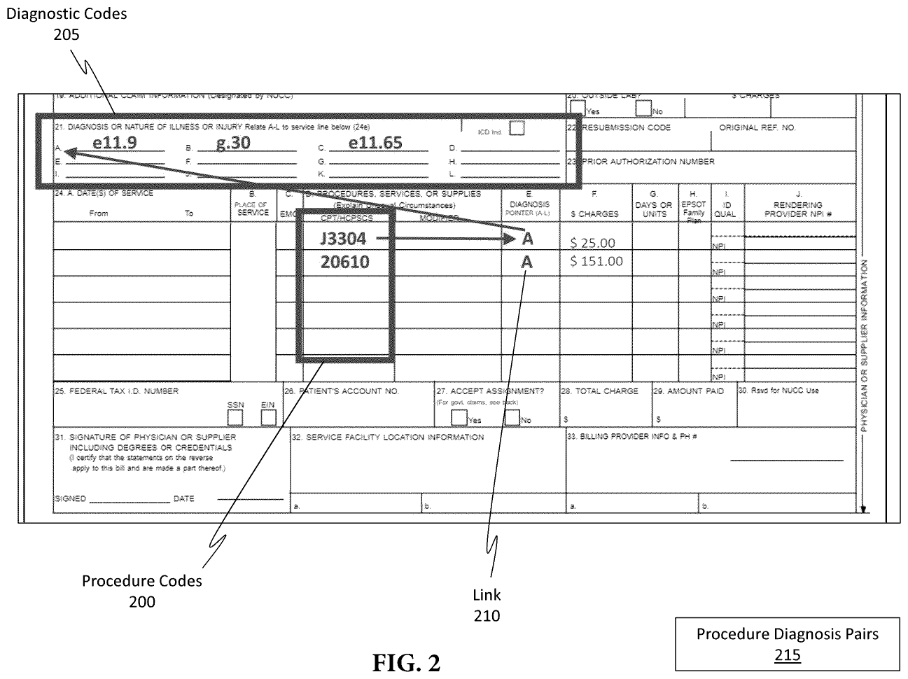

Having just described some of the benefits of the disclosed embodiments, attention will now be directed to FIG. 1, which illustrates an example of a so-called “professional” claim form 100. As shown in FIG. 2, the professional claim form includes a set of procedure codes 200 describing which procedures a patient was treated with. The professional claim form further includes a set of diagnostic codes 205. The diagnostic codes 205 describe what diagnosis a patient was given, such as perhaps the patient was diagnosed with diabetes. The procedures that were performed (as described by the procedure codes 200) can be linked to the diagnosis. For instance, the link 210 shows how the procedure code J3304 was performed for the diagnosis e11.9. In this manner, the professional claim form includes various procedure diagnosis pairs 215.

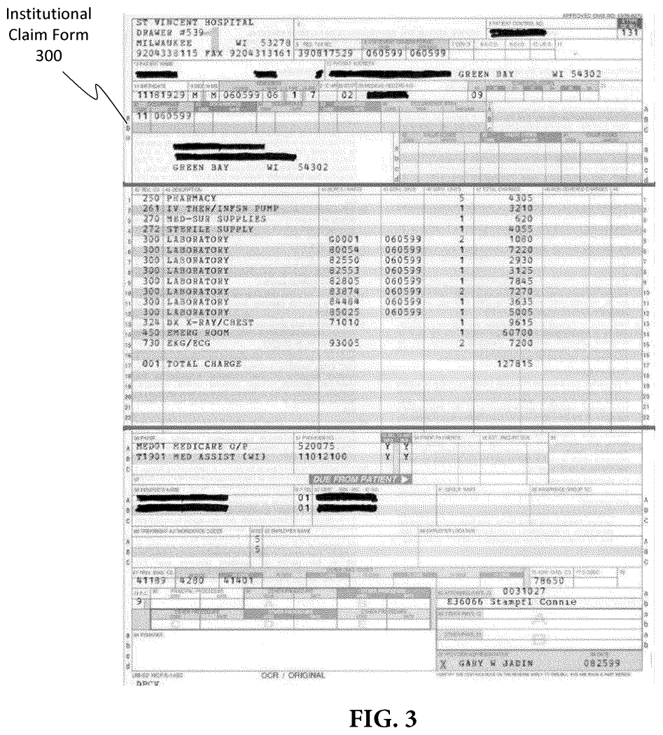

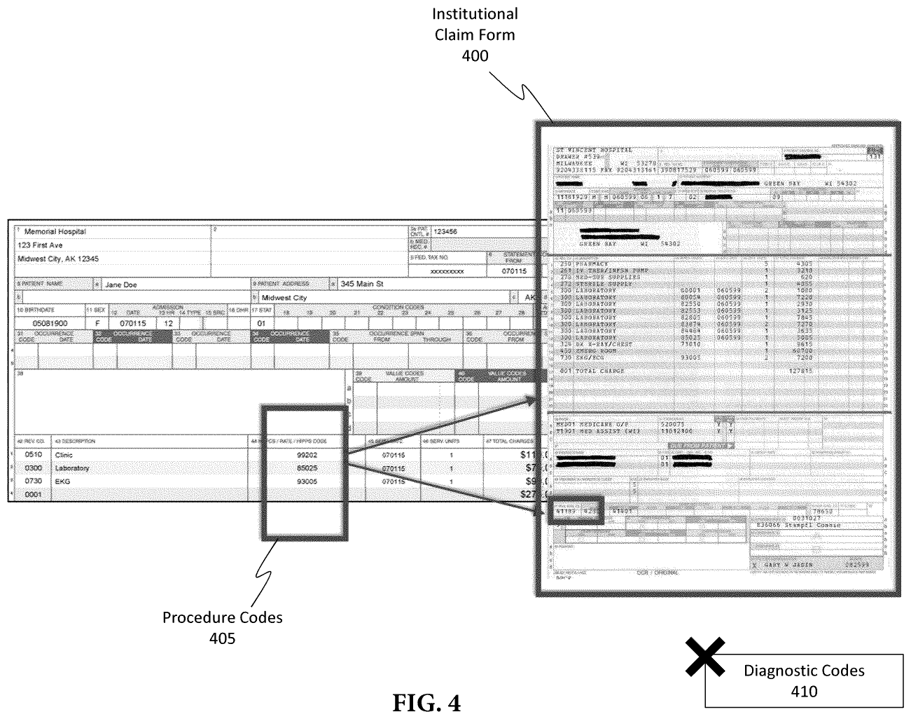

FIG. 3 shows another type of claim form, namely an institutional claim form 300. FIG. 4 shows the same institutional claim form 400 and further shows how the institutional claim form 400 includes procedure codes 405, similar to those that were described in FIG. 2. Notably, however, the institutional claim form 400 does not include diagnostic codes 410, as shown by the black “X” in FIG. 4.

As described above, there are generally two different types of claim forms, namely, the professional claim forms and the institutional claim forms. The professional claim forms generally include more specific information than that which is found in the institutional claim forms because the professional claim forms link specific procedures to specific diagnoses whereas the professional claim forms include only the procedure codes.

The disclosed embodiments are able to perform big data analysis on any type of claim form in order to generate an index for a specific medical condition. In fact, millions or even billions of claim forms and claims can be analyzed to extract relevant information for generating the index. The claim forms described in FIGS. 1-4 are exemplary of the types of forms that are used by the embodiments to generate the index.

### Example Methods and Architectures

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

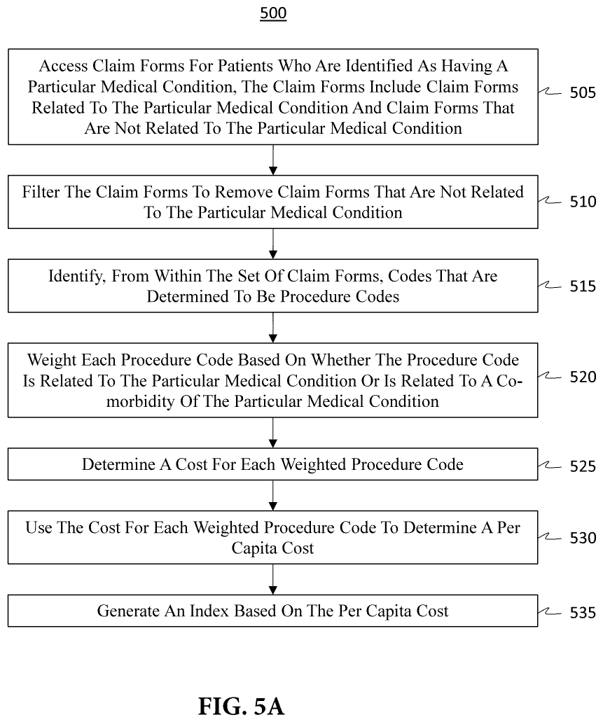

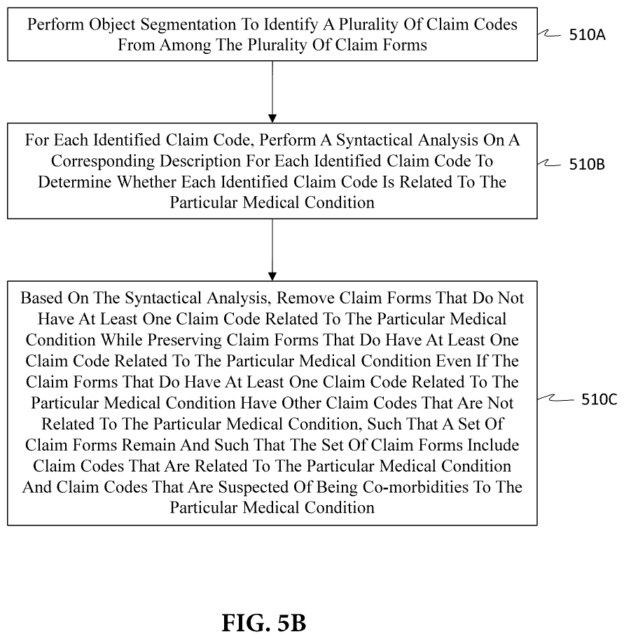

Attention will now be directed to FIGS. 5A and 5B, which illustrate a flowchart of an example method 500 for generating an index that is statistically sensitive to a cost of treating a medical condition. The medical condition can be any type of medical condition. A substantial portion of this disclosure will use diabetes as an example. One will appreciate, however, how diabetes is just being used herein for example purposes only, and the disclosed principles can be applied to any type of medical condition.

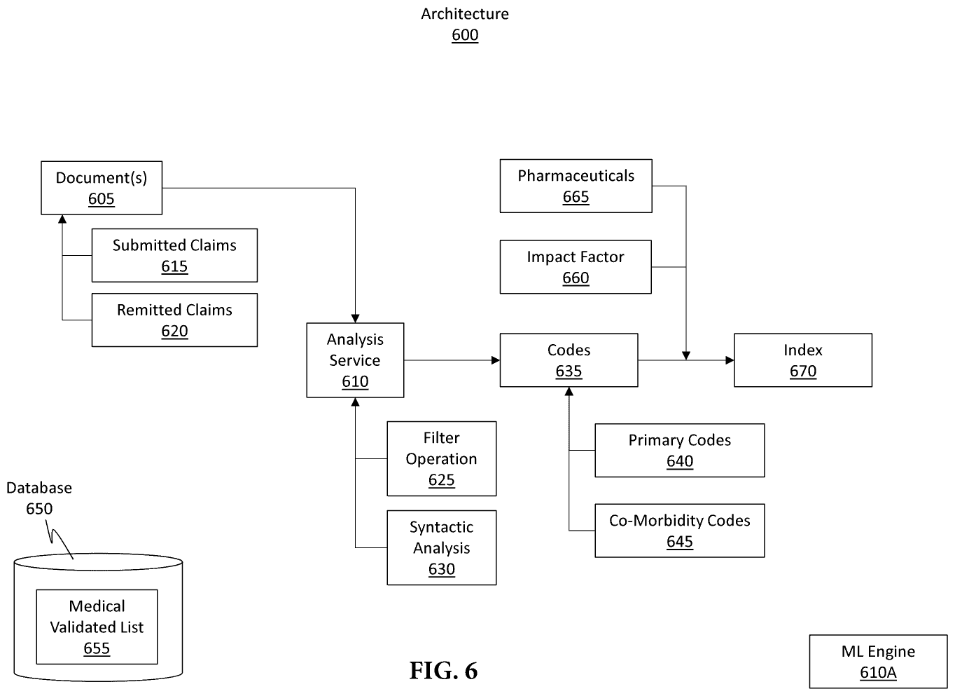

Method 500 can be implemented within the architecture 600 of FIG. 6. So, frequent reference will be made between the acts of FIGS. 5A and 5B as well as the components described in FIG. 6.

Method 500 includes an act (act 505) of accessing a plurality of claim forms for patients who are identified as having a particular medical condition. The plurality of claim forms include claim forms related to the particular medical condition and claim forms that are not related to the particular medical condition.

For instance, the claim forms for all diabetes patients over a specified time period (e.g., perhaps 1 day, 1 week, 1 month, 1 year, five years, or any other time period) can be obtained from a healthcare provider. Of course, these forms may be anonymized to protect the patient data. Often, a diabetes patient may submit a claim for something that is not related to diabetes. Thus, as act 505 mentions, the claim forms include forms that do relate to the medical condition (e.g., diabetes) and claim forms that do not relate to the medical condition (e.g., perhaps a diabetic person broke his/her arm).

With reference to FIG. 6, the architecture 600 shows how a set of document(s) 605 are fed as input into an analysis service 610 (or simply “service”). The set of document(s) 605 can be claim forms. Those claim forms can include submitted claims 615 and remitted claims 620. The submitted claims 615 are claims that have been submitted but that may not have been paid for (as of yet). The remitted claims 620 are claims that have been submitted and paid for, such that the remitted claims 620 describe the costs for actual claims.

The analysis service 610 can be a local service that is executing locally on a computer device. Alternatively, the analysis service 610 can be a cloud computing service that is executing in the cloud. In some cases, the analysis service 610 is or includes a machine learning engine 610A.

As used herein, reference to any type of machine learning may include any type of machine learning algorithm or device, convolutional neural network(s), multilayer neural network(s), recursive neural network(s), deep neural network(s), decision tree model(s) (e.g., decision trees, random forests, and gradient boosted trees) linear regression model(s), logistic regression model(s), support vector machine(s) (“SVM”), artificial intelligence device(s), or any other type of intelligent computing system. Any amount of training data may be used (and perhaps later refined) to train the machine learning algorithm to dynamically perform the disclosed operations.

The ML engine 610A, and thus the analysis service 610, can perform big data analysis on the document(s) 605. In some embodiments, the analysis service 610 can perform image or object segmentation on the document(s) 605 to identify specific types of information, such as perhaps codes, as will be described in more detail shortly.

Method 500 includes an act (act 510) of filtering the plurality of claim forms to remove claim forms that are not related to the particular medical condition. The filtering process is described in FIG. 5B. Specifically, the filtering process includes an act (act 510A) of performing image or object segmentation to identify a plurality of claim codes from among the plurality of claim forms. For instance, if the document(s) are pdf documents, the ML engine can analyze the document portions via image or object segmentation to extract the portions of the document(s) related to medical codes.

For each identified claim code, act 510B includes performing a syntactical analysis on a corresponding description for each identified claim code to determine whether each identified claim code is related to the particular medical condition. It is typically the case that each claim code is described in detail via a medical validated list of claim codes. The list or description can be reviewed by the analysis service to parse out text to determine whether a code is related to a particular medical condition. By “related,” it is generally meant that the code's description describes medical aspects that would indicate the patient has the particular medical condition. Further details on this aspect will be provided later.

Based on the syntactical analysis, act 510C of FIG. 5B includes removing claim forms that do not have at least one claim code related to the particular medical condition while preserving claim forms that do have at least one claim code related to the particular medical condition even if the claim forms that do have at least one claim code related to the particular medical condition have other claim codes that are not related to the particular medical condition. For instance, a claim form might have three codes included therein. The first code might be related to diabetes. For example, the claim code's corresponding description might say something like the following: “insulin issues related to diabetes.” The second claim code might be related to hypertension. The third claim code might be for disorders related to lipid metabolism. Hypertension and lipid metabolism disorders are directly linked to diabetes, though they are common co-morbidities to diabetes. As described above, this claim form will be retained because it includes at least one code corresponding directly to diabetes.

As a result, a set of claim forms remain, and the set of claim forms include claim codes that are related to the particular medical condition and claim codes that are suspected of being co-morbidities to the particular medical condition. Continuing with the above example, the first claim code is directly linked to diabetes while the other two claim codes are related to conditions that are suspected of being co-morbidities to diabetes.

FIG. 6 shows how the analysis service 610 is able to perform a filter operation 625 and a syntactic analysis 630. The analysis service 610 can perform object or image segmentation on the claim forms (e.g., the professional claim form 100 of FIG. 1 and/or the institutional claim form 400 of FIG. 4) to identify any available diagnostic codes and procedure codes.

Returning to FIG. 5A, act 515 includes identifying, from within the resulting set of claim forms, codes that are determined to be procedure codes (as compared to diagnostic codes). For instance, the procedure codes 405 from FIG. 4 can be identified. With reference to FIG. 6, the analysis service 610 can identify the codes 635. These codes can include primary codes 640, which are codes that are linked with the medical condition, and co-morbidity codes 645, which are codes that are linked with a co-morbidity to the medical condition. Further details on this aspect will be provided later.

As mentioned previously, the embodiments perform a syntactic analysis to determine whether a code is related to a medical condition. To do so, the analysis service 610 can consult a database 650 comprising a medical validated list 655 that provides a textual description of what each code represents. The embodiments can parse and analyze the medical validated list 655 to determine whether a code is related to a particular medical condition. As an example, suppose a claim form includes the code “eff54.” The service can query the medical validated list 655 to obtain the textual description associated with that code. If the textual description reads something like: “insulin issues related to pancreas,” then the embodiments can parse the text and determine whether the text is related to a medical condition, such as perhaps diabetes.

Here, even though the text does not explicitly mention the term “diabetes,” the embodiments can perform an intelligent analysis on the text to determine that the terms “insulin” and “pancreas,” especially when used together, are likely related to diabetes. Thus, an intelligent review of the text can enable the embodiments to determine whether a code is related to a particular medical condition. Of course, some textual descriptions will include the name of the medical condition.

Act 520 of FIG. 5A includes weighting each procedure code based on whether the procedure code is related to the particular medical condition or is related to a co-morbidity of the particular medical condition. The weighting is performed by applying an impact factor to the procedure codes. Notably, the impact factor is defined as a number of times that the procedure code is identified as being associated with a diagnostic code for the particular medical condition within the set of claim forms divided by a total number of times that the procedure code is identified within the set of claim forms.

FIG. 6 shows how the embodiments can incorporate the impact factor 660 to determine which procedural claims are primarily related to the actual medical condition and which claims are primarily related to a co-morbidity.

Act 525 of FIG. 5A then includes determining a cost for each weighted procedure code. The cost is then used (act 530) for each weighted procedure code to determine a per capita cost. Finally, an index is generated (act 535) based on the per capita cost. In some cases, as shown in FIG. 6, the cost for pharmaceuticals 665 can also be incorporated in order to generate the index 670.

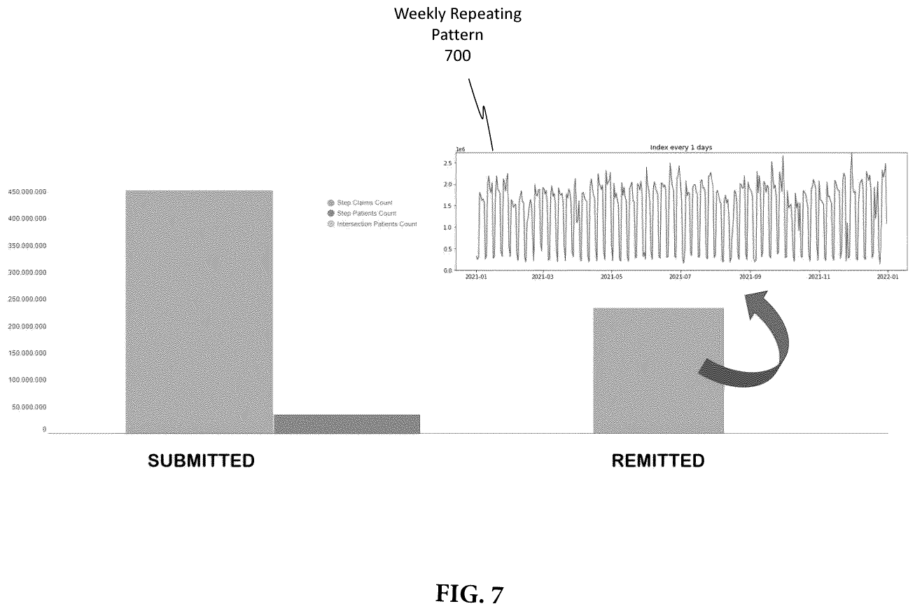

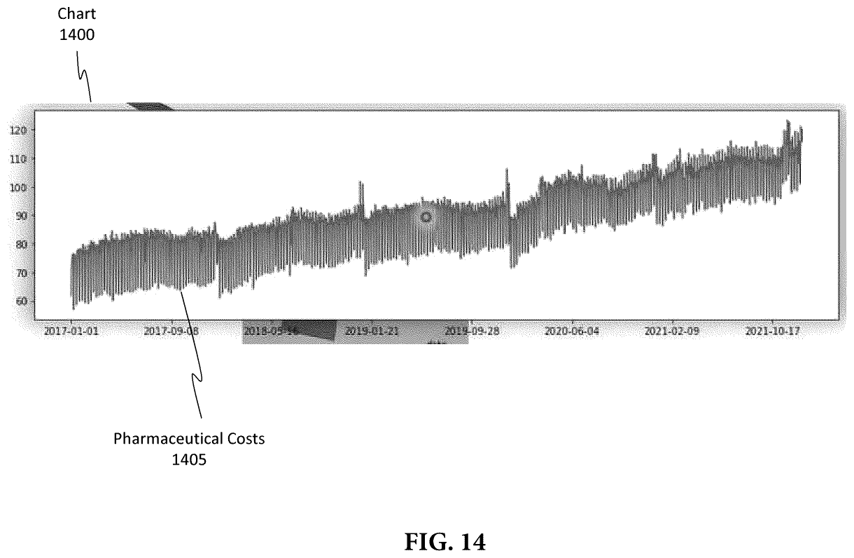

Accordingly, having just described some of the disclosed principles in a broad manner, it will now be useful to provide a specific example. FIGS. 7 through 18 will thus provide the framework for a specific example related to diabetes, and the generation of an index for that disease.

### Specific Example Scenario

As described previously, the embodiments are designed to create an index that is statistically sensitive to the cost of treating any particular medical condition. First, the embodiments extract relevant data from the claim forms, as mentioned previously. To do so, the embodiments focus on the different diagnostic codes that, in one way or another point, to the existence of a medical condition.

Some of the claim forms show or list procedures or services that were administered to the patient to treat the list of diagnoses. Some of those forms also have links to connect each procedure with the specific diagnostic code that requested it. Using these different codes, the embodiments can assess the cost of diagnostic codes by adding the cost of its associated procedures.

Regarding the calculation of the index itself, as indicated above, the embodiments first obtain the claim form data from a vendor. For example, the embodiments can retrieve all the claims that were made by diabetic patients in a certain period of time. The result of such a retrieval operation is that all the claims of patients with diabetes will be returned, including claims that are completely unrelated to diabetes (e.g., because patients with diabetes can still make claims for other diseases).

In a specific example, five years of diabetes patient data was obtained. Throughout these five years, the use case scenario ingested data for about 30 million patients and about two billion claims. Out of those two billion claims, however, the service is interested in only claims that have at least one diagnostic code pointing to diabetes. So, claims that have nothing to do with diabetes are removed or filtered, as described previously.

The strategy to identify a claim that is related to diabetes is based on the codes embedded in the claim forms. Claim forms that have at least one diabetes code are identified. This identification process is based on a syntactic analysis of the description of the codes, code by code and claim by claim.

More specifically, the disclosed service checks the description of every code in every claim. If the service finds at least one code description that is syntactically close to diabetes, then that claim is kept for further analysis. Otherwise, the claim is removed because the service did not find any description that was syntactically close to diabetes. This analysis process is repeated for all the claims in the set, thereby resulting in a set of claims that are all related to diabetes. Accordingly, for claims that have at least one code related to diabetes, the service preserves those claims and all its codes, even if that claim includes other, non-diabetes related claims.

This strategy is beneficial because it allows the resulting index to consider co-morbidities and not merely diabetes codes. Co-morbidities will be represented by those diagnostic codes that do not necessarily point to diabetes but that are often still included in the same claim form as codes that do point to diabetes. In other words, the codes that accompany the codes for diabetes within a claim form are typically at least suspected of being a co-morbidity. Notably, the cost of treating a condition like diabetes is often heavily influenced by the cost of its co-morbidities, so configuring the index to include the costs for the co-morbidities is highly beneficial. In some cases, the data can be further segmented based on patient demographics.

It is also beneficial to recognize that the identified claims might be so-called “submitted” claims, meaning that they do not yet bear any actual cost; rather, they just reflect the amount of money that the healthcare provider intends to collect from the payer. What the healthcare insurance actually pays is accessible only in so-called “remitted” claims. In this sense, the remitted data is basically the corrected version of the submitted claims. It might be the case, however, that the service does not have remitted data for every submitted claim. In fact, it is typically the case that only about 50% of remittance for all of our submitted data can be obtained.

One benefit of some of the disclosed indexes is that (some) indexes might not strictly reflect an actual cost, but rather the indexes might emphasize the changes or fluctuation in the cost, which fluctuations are often more beneficial than the actual cost. That being said, some embodiments do focus on the actual cost and thus use the remitted data to keep the index as realistic as possible and arguably more sensible to subtle changes.

Initially, the service first attempts to measure the daily cost of all the procedures in the remitted claims. FIG. 7 is illustrative.

FIG. 7 shows an isolated pattern that repeats itself every certain period of time, as shown by the weekly repeating pattern 700. This pattern is the effect of the weekends in the volume of medical visits, and therefore medical claims. That is, every seven days, as shown, there is a reduced number of claims. This drop occurs because the cost of medical procedures or services drops during the weekends.

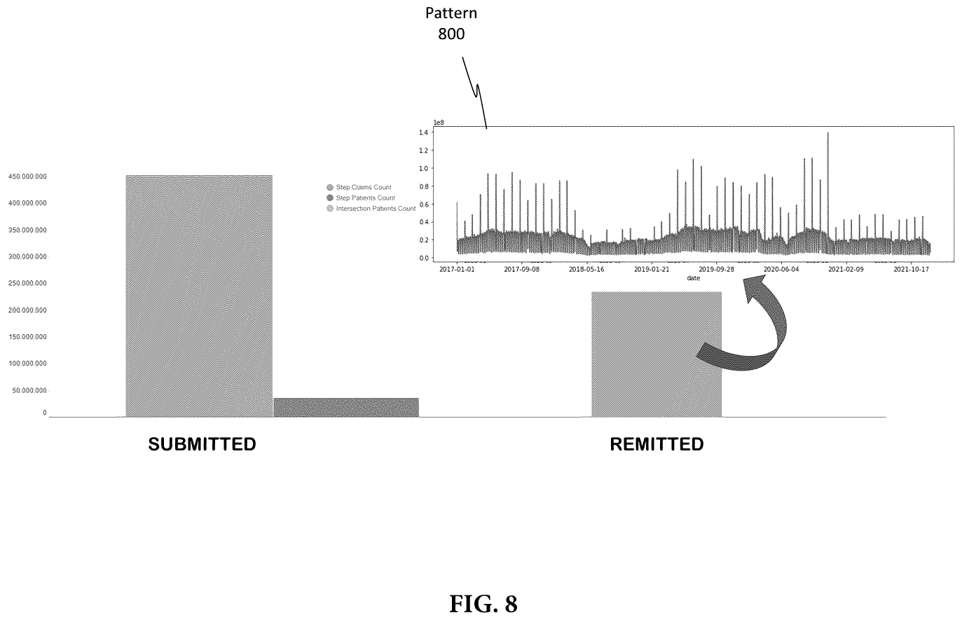

FIG. 8 shows a larger scale illustration. As shown in FIG. 8, one can also observe some spikes rising every first day of the month, as shown by the monthly repeating pattern 800. This second pattern is due to the accumulation of costs. Some procedures or services are charged monthly, such as administrative fees, private nursing, private rooms, monthly treatments, and so on. It is beneficial to further refine the data because it might be the case that the data is including the cost of some procedures that have actually little connection with diabetes and therefore their costs should have less impact in the cost of diabetes as a whole.

Recall that during the initial pruning or filtering of the claims, the service kept all the codes in those diabetes-related claims, even codes that were not related to diabetes or a co-morbidity of diabetes. So, many of those codes and their associated procedures may have nothing to do with diabetes or co-morbidities. As such, it is beneficial to further refine the data to eliminate this extraneous impact.

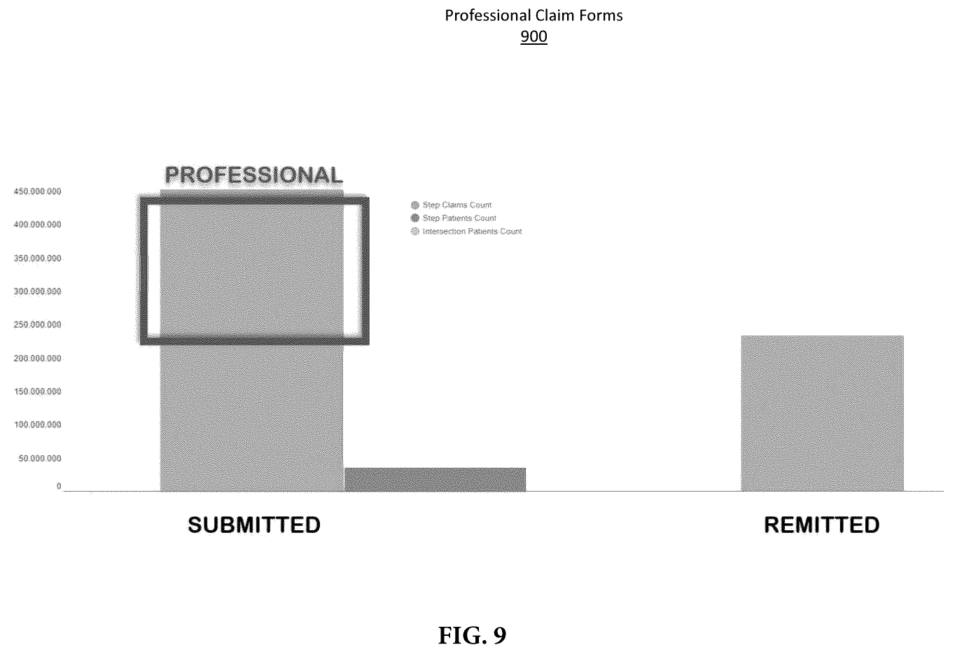

Further recall, for institutional claims, the service was not able to parse a linkage between procedural codes and diagnostic codes. FIG. 9 shows how the professional claim forms 900 (i.e. those with the linkage) comprise only a portion of the total number of claim forms that are being assessed.

Therefore, every time a procedural code is extracted from institutional claim forms, there is an uncertainty as to what disease that procedure was performed for. That is, the service is not certain if the procedure was requested by a diagnostic code related to diabetes by a co-morbidity or perhaps to some other unrelated condition. It is desirable to not allow that code to impact the disclosed index. Thus, the service is configured to perform some additional operations in an effort to remove the effects of those codes.

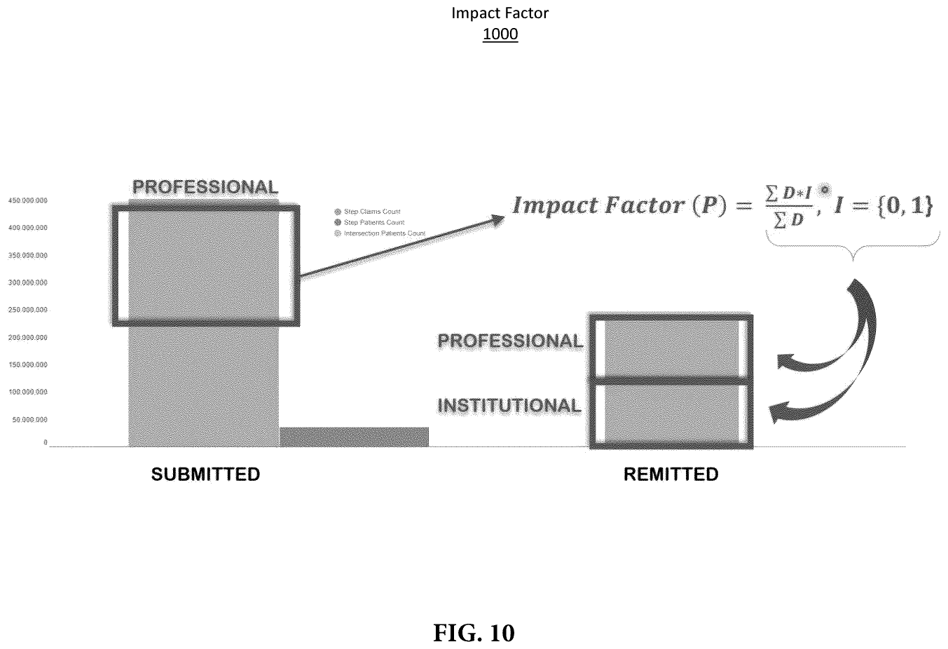

To do so, the service relies on an impact factor, as shown by the impact factor 1000 of FIG. 10. This impact factor weights the cost of each procedure according to the relevance of the procedure to the medical condition of interest (e.g., diabetes) or to that condition's co-morbidity.

To create this impact factor, which quantifies the relationship between procedures and diagnostic codes, the service uses the data that provides examples of relationships. That is, to generate the impact factor, the service relies on the professional claim forms, which show linkages between procedures and diagnoses.

The service uses the professional claim forms to train a model to learn the relationship states. Later, this relationship knowledge can be projected to the institutional claim forms where there was no identifiable relationship.

The impact factor can be defined for any particular procedure. The impact factor is defined as a number of times that a procedure code is identified as being associated with a diagnostic code for a particular medical condition (e.g., diabetes) within the professional claim forms divided by a total number of times that the procedure code is identified within the professional claim forms (it might be the case that the procedure is associated with a different, non-diabetes related diagnostic code). To illustrate, the bottom summation in FIG. 10 stands for the number of all diagnoses that requested a service for the procedure. The top summation is similar except for the additional weighting factor (“I”). Here, each diagnosis is weighted by either a zero or a one depending on whether the diagnosis is related to diabetes or any co-morbidity, as determined by the diagnostic code.

The service identifies co-morbidities by consulting a medical validated list of typical co-morbidities. The service can look up the codes in the list to determine whether a code is related to a co-morbidity. Additionally, the service can count the number of times that a code that is not a diabetes code appears in a diabetes claim form. The codes with the highest number of appearances can be determined to be a code for a co-morbidity. This is how the service shapes the impact factor for all procedures. The service can also utilize this process in the remitted data for both professional and institutional claim forms. In this manner, the service can beneficially transfer knowledge from one type of claim form (e.g., the professional claim form) to another type of claim form (e.g., the institutional claim form).

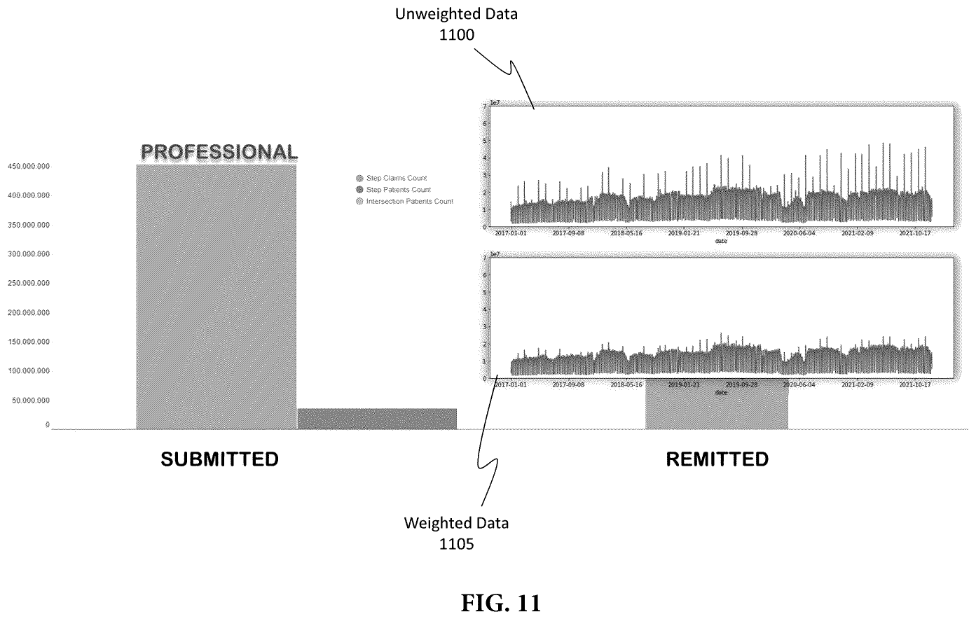

The results of the service weighting the data using the impact factor are shown in FIG. 11. In particular, FIG. 11 shows the unweighted data 1100, which includes numerous spikes, and the weighted data 1105, which is a much flatter function because the monthly spikes now have less impact due to their disconnection or their little direct relation with diabetes treatment.

The weighted data 1105 represents the total daily cost of procedures related to diabetes. It is often desirable, however, to obtain a patient centered depiction. To achieve that, the service can divide the weighted data 1105 by the number of patients that are consuming those procedures daily.

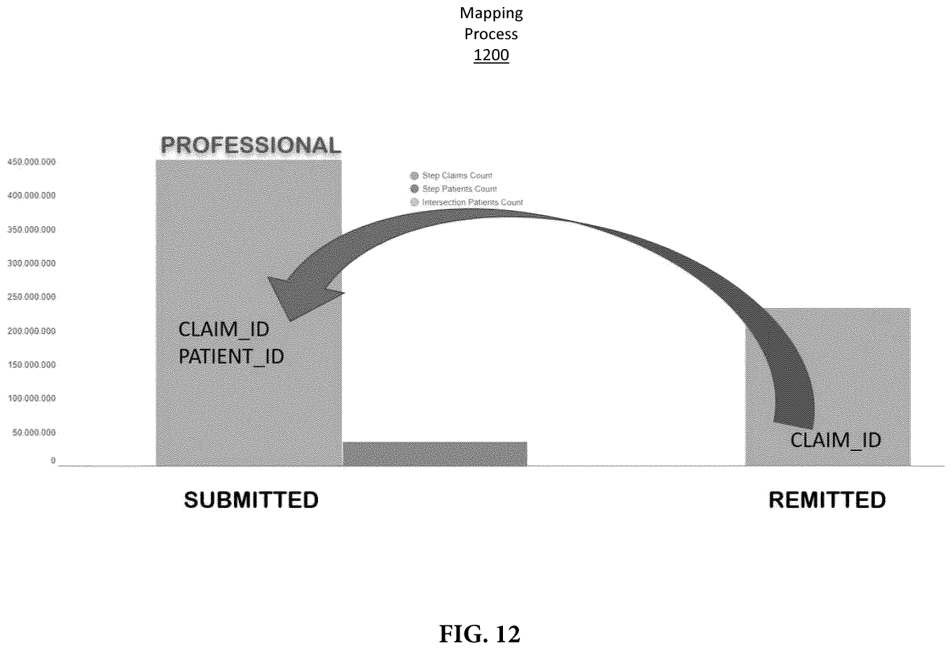

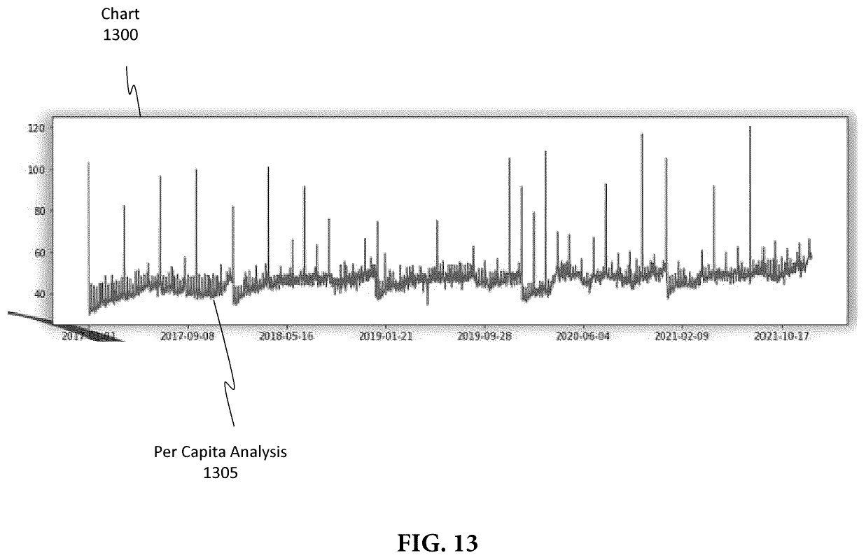

The remitted data typically does not have information about patients. In some scenarios, the service maps the remitted data back into the submitted claim data on a claim by claim basis in order to find the claimer/patient that filed the claim and in order to perform a per capita analysis. FIG. 12 showing the mapping process 1200 for mapping the remitted data back to the submitted data. FIG. 13 shows a chart 1300 reflecting the resulting per capita analysis 1305 based on the above described operations.

Apart from just medical claims, it is also often beneficial to factor in the costs associated with pharmaceutical claims. Generally, pharmaceutical claims are easier to process because there is no professional claim form versus institutional claim form nor is there remitted claims versus unremitted claims. The disclosed service is able to account for drugs that patients are consuming, including drugs for a primary medical condition (e.g., diabetes) and drugs for co-morbidities.

Accordingly, the service processes the cost of these pharmaceutical claims, as shown by the chart 1400 showing the pharmaceutical costs 1405 for a particular medical condition (e.g., diabetes). Some embodiments adopt an impact factor that quantifies the relationship between a particular medicine and a particular medical condition or any co-morbidity as well. As such, the embodiments can choose to create this impact factor by identifying the most consumed or the most popular drugs among the patient population having the specific medical condition. Those drugs are likely to be the ones intended to treat the medical condition or the co-morbidities. The price of a particular medicine will be weighted in the index according to its popularity, so the more popular the medicine is among the class of patients, the more weight will be assigned to that drug.

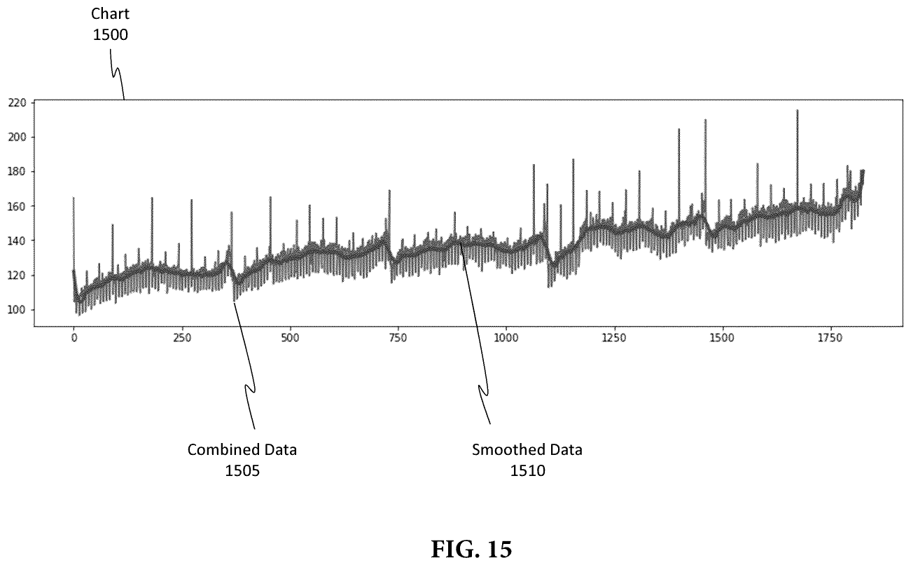

The pharmacy component of treating a medical condition can then be added to the procedure component already calculated. These two aspects are then combined, as shown by the chart 1500 of FIG. 15, which shows a combination of the procedural costs and the pharmaceutical costs, as represented by the combined data 1505. Optionally, a smoothing function can be applied to the combined data 1505 to generate the smoothed data 1510. The smoothed version captures the principal trends and tendencies of the costs associated with a particular medical condition. Using the smoothed data 1510 also allows the service to avoid local oscillations that generally mean nothing in a larger time scale.

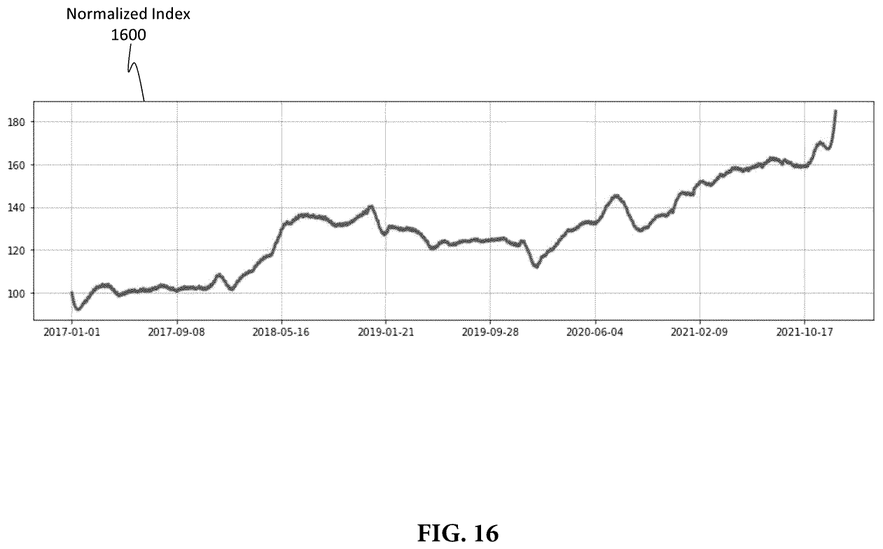

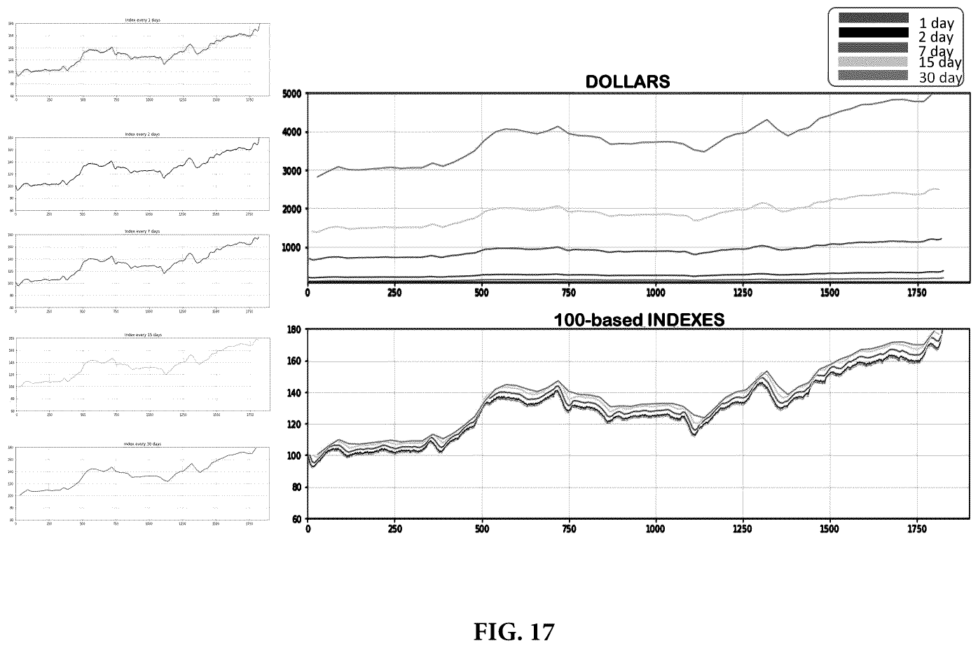

FIG. 16 shows how the index looks when passed from cost in dollars to a normalized scale, as shown by the normalized index 1600. FIG. 17 shows the index in different time scales (the top graph). When combined together (the bottom graph), the different time scaled versions of the index generally align with one another.

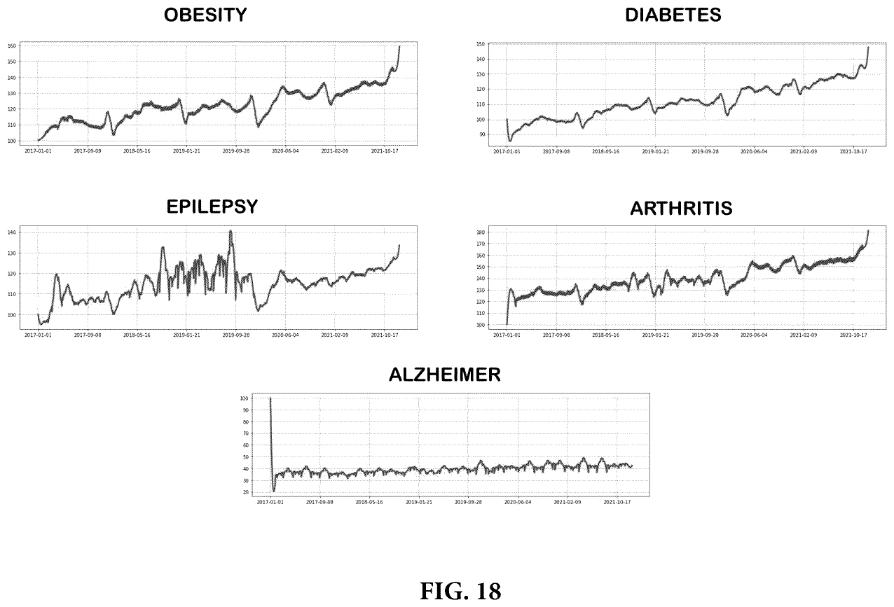

FIG. 18 shows some other indexes that can be created for some other medical conditions. Such conditions include the costs associated with obesity, epilepsy, arthritis, and Alzheimer's. Accordingly, the disclosed embodiments can beneficially generate an index for a particular medical treatment, where the index generally reflects the costs associated with that medical condition. By generating this index, a futures market for the healthcare industry can be generated, and healthcare costs can be stabilized.

### Generating an Index for Medical Expenses

To be a highly robust and accurate index, the index can be tailored to not only track the costs related to a service for treating a medical condition but it should also track the costs related to pharmaceuticals that are used to treat the medical condition. The above description was primarily focused on generating an index associated with the service expenses. The following description will now focus on generating an index associated with the drug expenses. One will appreciate how the combination of these two indexes can allow a futures market to be generated and can help stabilize the healthcare industry.

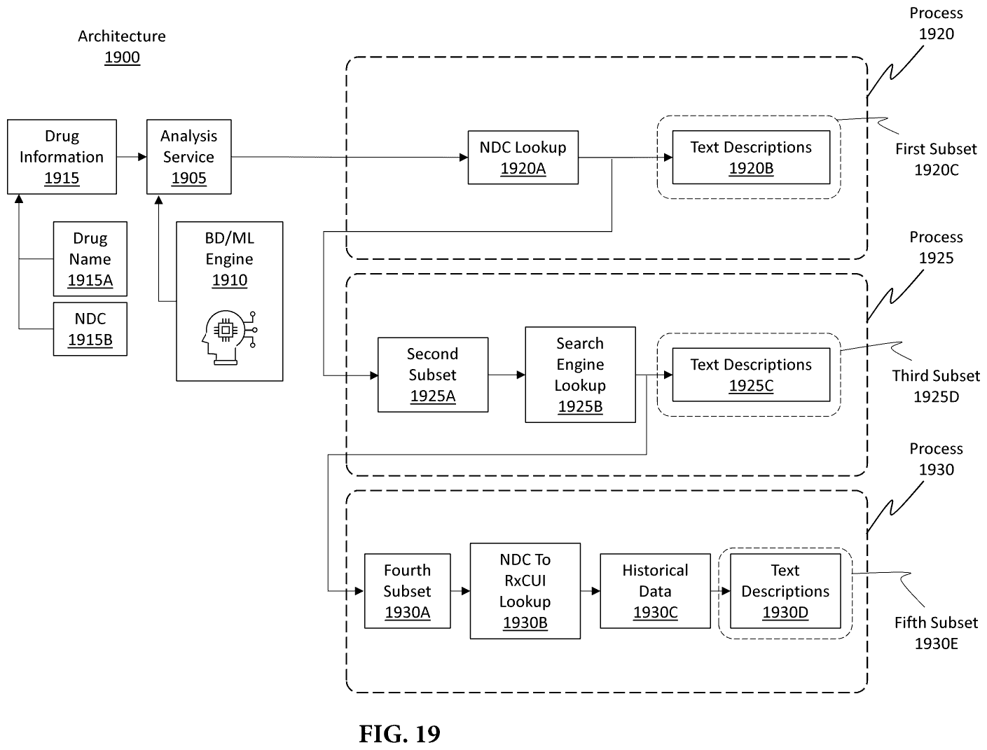

Attention will now be directed to FIG. 19, which illustrates an example architecture 1900 that is configured to perform the disclosed operations. Architecture 1900 is shown as including an analysis service 1905. The analysis service 1905 can be a local service operating on a local device or, alternatively, the analysis service 1905 can be a cloud service operating in the cloud. In some cases, the analysis service 1905 can be a hybrid that perhaps includes a client service operating on a local device and a cloud-based service. Optionally, the analysis service 1905 can be the same as the analysis service 610 of FIG. 6.

In some implementations, the analysis service 1905 includes or implements a big data and machine learning (BD/ML) engine 1910. As used herein, reference to any type of machine learning may include any type of machine learning algorithm or device, convolutional neural network(s), multilayer neural network(s), recursive neural network(s), deep neural network(s), decision tree model(s) (e.g., decision trees, random forests, and gradient boosted trees) linear regression model(s), logistic regression model(s), support vector machine(s) (“SVM”), artificial intelligence device(s), or any other type of intelligent computing system. Any amount of training data may be used (and perhaps later refined) to train the machine learning algorithm to dynamically perform the disclosed operations.

The analysis service 1905 receives as input any type of drug (aka pharmaceutical) information 1915. In some cases, the drug information 1915 is provided in the form of one or more claim forms. For instance, a claim form can be provided as a pdf or as some other document format. Optionally, the analysis service 1905 can perform optical character recognition on the forms to detect information related to drugs.

The drug information 1915 can be provided in other ways as well, such as via access to a database that stores the drug information 1915. In some cases, the drug information 1915 can even be entered by a user manually. Regardless of how the drug information 1915 is accessed, that drug information 1915 is provided as input to the analysis service 1905. One will appreciate how any number of drugs can be used. Often, the number of drugs that are analyzed by the analysis service 1905 is in the hundreds of thousands, though more or less can be used.

The drug information 1915 typically includes at least a name of the drug or drugs, as shown by drug name 1915A. The drug name 1915A identifies the scientific name of the drug and perhaps the market recognized name of the drug. In some cases, the drug information 1915 also includes the national drug code (NDC) 1915B for the drug. In some cases, the analysis service 1905 uses the drug name 1915A to execute a query to identify that drug's corresponding NDC 1915B. It should be noted that the architecture 1900 is structured to operate in a manner so as to recognize or identify any number of drugs. Thus, the number of drugs that can be processed and analyzed by the analysis service 1905 is essentially unlimited.

Having acquired the NDC 1915B for any number of drugs (e.g., perhaps hundreds of thousands of drugs), the analysis service 1905 can then use the NDC 1915B and the drug name 1915A to perform a number of different processes. In some cases, these processes are performed in parallel with one another. In some cases, these processes are performed in serial, one after the other. FIG. 19 illustrates these processes as process 1920, process 1925, and process 1930. Although FIG. 19 shows a generally serial relationship between process 1920, 1925, and 1930, as mentioned above, one will appreciate how the various processes can optionally be performed in parallel with one another. Information that is obtained in response to performing one processes can be used to supplement or augment any information that is obtained in response to performing one or more of the other processes. Thus, one process can be used to add on to any existing information that is discovered as a result of performing another process.

Process 1920 involves the analysis service 1905 performing an NDC lookup 1920A operation via a website query in an attempt to identify text descriptions 1920B. That is, there are various online resources that are available and that provide a mapping between a drug's NDC and a textual description for that drug. As used herein, the phrase “textual description” refers to a written description that describes what medical conditions the drug can be used for and, optionally, what compositions are included in the drug.

The analysis service 1905 is able to navigate to these online sources, repositories, or databases and use the NDC 1915B to execute a query against that source in an effort to identify the textual description for the drug associated with the NDC 1915B. In some cases, pre-generated or pre-structured queries can be compiled, where those queries are structured for execution against a particular online resource. For instance, the structure of the online resource may be known beforehand, and a number of queries can be generated based on that known structure.

In some cases, the queries are generated in a dynamic manner. For instance, the analysis service 1905 can navigate to the online resource and then dynamically identify that resource's structure. Based on this learned information, the analysis service 1905 can then tailor or generate queries that are executable in accordance with this learned structure.

If a first online source or resource does not have the requested textual description, then the NDC 1915B can optionally query one or more additional online sources in an attempt to identify the written description. An example of an online source can be the National Institute of Health (NIH) website. Another example of an online source can be the National Library of Medicine. Yet another example of an online source can be the National Drug Code Directory.

In some cases, the analysis service 1905 may be tasked with querying a threshold number of online sources before the analysis service 1905 resorts to a different mechanism (process) for identifying the textual description for that drug.

As mentioned previously, the analysis service 1905 can perform this operation for any number of drugs. As an example, suppose the analysis service 1905 is tasked with identifying the textual description for 300,000 different drugs.

A result of executing the website query (e.g., for the 300,000 drugs) returns a first set of text descriptions 1920B for a first subset of pharmaceuticals, as shown by first subset 1920C. By way of non-limiting example, suppose the analysis service 1905 was able to successfully identify textual descriptions for 200,000 of the 300,000 drugs. The first subset 1920C thus includes 200,000 drugs and corresponding textual descriptions. The remaining 100,000 drugs are then included in a second subset 1925A.

Before continuing the description with regard to the second subset 1925A, it is worthwhile to note that the analysis service 1905 is configured in an intelligent manner so as to avoid situations where the online resource might consider the analysis service 1905 to be a malicious entity. For instance, when online resources detect a large amount of network traffic from a particular entity, an online resource may consider that entity as one that is targeting the online resource for a denial of service (DOS) attack or some other attack. In an attempt to mitigate such scenarios, the analysis service 1905 can optionally use a dynamic Internet Protocol (IP) address.

In some cases, the analysis service 1905 may change its dynamic IP address after a threshold number of requests/queries are submitted to the online resource. The threshold number can be set to any value. In some cases, the threshold number is chosen to be the same number each time the IP address is changed.

On the other hand, the threshold number can be randomly chosen each time the IP address is changed. As an example, during a first iteration, the threshold may be set to 97 query requests. After the 97th query request is submitted, the analysis service 1905 can not only change its IP address but it can also change the threshold to a new value, such as perhaps 45. After the 45th request is submitted, the analysis service 1905 can again change its IP address and can again change the threshold number, such as perhaps to 87. The threshold number can optionally be based on a formula or it can be chosen at random. By changing the IP address, the analysis service 1905 can avoid a situation where the online resource may view the analysis service 1905 as a malicious attacker.

With the second subset 1925A (e.g., the 100,000 remaining drugs for which textual descriptions were not found as a result of performing process 1920), the analysis service 1905 may then perform the process 1925.

Process 1925 generally includes the analysis service 1905 using the names of the drugs in the second subset 1925A to perform a search engine lookup 1925B operation, such as perhaps using Google, Bing, or any other search engine. The analysis service 1905 uses the names of the drugs in the second subset 1925A as parameters in various search engine queries.

A result of executing the search engine queries returns a second set of text descriptions 1925C for a third subset 1925D of the pharmaceuticals (e.g., from the original 300,000). In an example scenario, the analysis service 1905 may have been able to successfully obtain textual descriptions for 75,000 drugs in the second subset 1925A (which included 100,000 drugs), such that the third subset 1925D includes 75,000 drugs and corresponding textual descriptions. The remaining 25,000 drugs are then included in a fourth subset 1930A.

The analysis service 1905 may then perform the process 1930 on the fourth subset 1930A of drugs (e.g., 25,000 remaining). The process 1930 generally involves causing the analysis service 1905 to use the NDCs for each pharmaceutical in the fourth subset 1930A as a parameter in a second website query, which is referred to as an NDC to RxNorm Concept Unique Identifier (RxCUI) lookup 1930B operation. Various online sources are available to use the NDC to lookup that drug's corresponding RxCUI.

A result of executing the second website query returns historical data 1930C comprising an RxCUI for each drug that is queried. The RxCUIs are subsequently used by the analysis service 1905 as parameters in a third website query, which can query various sources that include information for drugs based on those drugs' RxCUIs. A result of executing the third website query returns a third set of text descriptions 1930D for the drugs in the fifth subset 1930E.

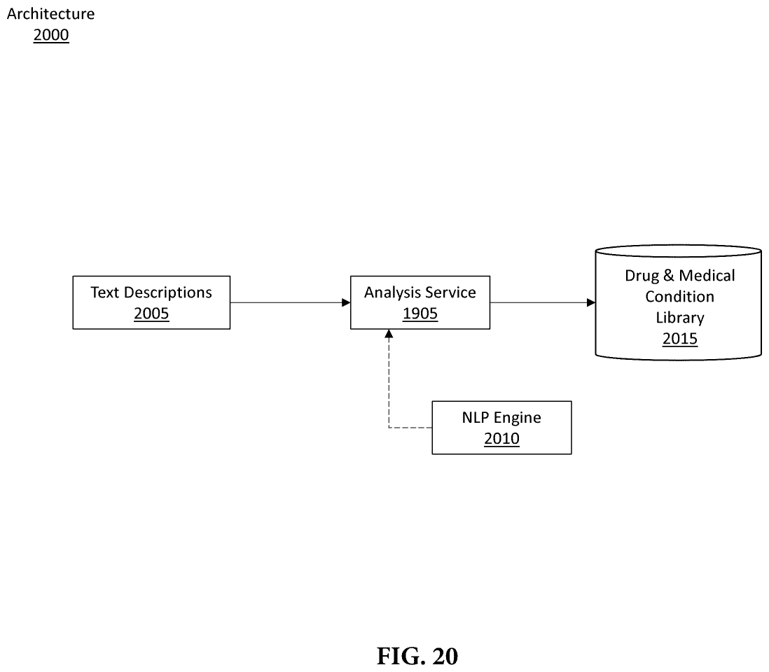

FIG. 20 shows an architecture 2000, which is a build-on, extension, or add-on to the architecture 1900 of FIG. 19. The analysis service 1905 compiles the text descriptions 1920B, 1925C, and 1930D from FIG. 19 to generate the text descriptions 2005. Each drug has its own corresponding text description.

In some implementations, the analysis service 1905 includes a natural language processing (NLP) engine 2010. In some cases, the NLP engine 2010 is a part of the BD/ML engine 1910 from FIG. 19. The NLP engine 2010 performs a semantic analysis on the text descriptions 2005 to identify correlations between a drug and what medical condition that drug is prescribed to treat. An example will be helpful.

Suppose the drug “Insulin Lispro” had the following textual description associated with it: “Insulin Lispro is used to help control high blood sugar in diabetic people.” The analysis service 1905 is able to parse out the different words in that textual description (e.g., using NLP processes). The analysis service 1905 can then analyze those words and determine that the drug “Insulin Lispro” is associated with the medical condition “diabetes.” From this identification, the analysis service 1905 can then form a linkage between the drug “Insulin Lispro” and the medical condition “diabetes.” This identified linkage or relationship can then be stored in the form of a drug and medical condition library 2015, which can be indexed and which can be searchable. In some cases, a specific medical condition might not be mentioned in a text description, but the analysis service 1905 can nevertheless make an informed or intelligent inference as to what medical condition the drug may treat.

As an example, suppose the previous text description was simply: “Insulin Lispro is used to help control high blood sugar.” Here, the analysis service 1905 can identify the phrase “high blood sugar.” Using that phrase, the analysis service 1905 can optionally execute a search query to determine what medical condition is associated with those key terms or parameters. From available literature, the analysis service 1905 can infer that high blood sugar is typically linked with diabetes. In some cases, a confidence metric can be assigned to the inference to reflect how sure or how confident the analysis service 1905 is with regard to its decision.

The analysis service 1905 can perform the above processes for any number of different drugs. As a result, the drug and medical condition library 2015 can include any number of identified relationships between drugs and medical conditions. In some cases, a drug may be linked with multiple different medical conditions. The drug and medical condition library 2015 can include multiple relationships for a single drug. In some optional cases, the medical conditions may be ranked, such as perhaps when a drug is primarily used for a first medical condition and can also be a supplemental medication for a second medical condition. By building up this library, users are highly benefitted because those users can then query this single source of information to discover what medical conditions a drug is used for instead of having to query potentially multiple different sources.

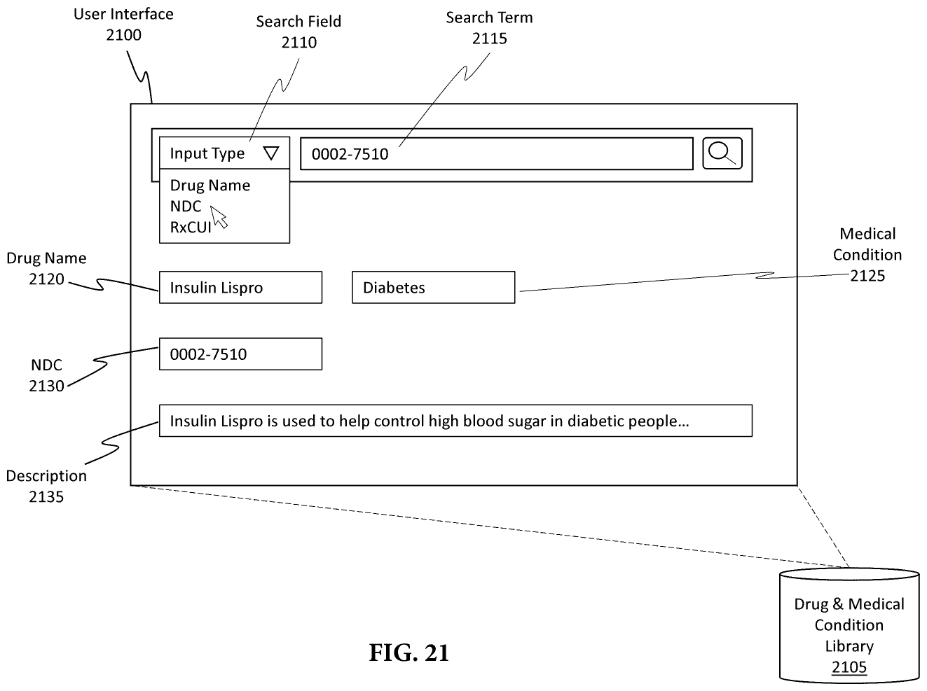

FIG. 21 shows an example user interface 2100 that can be used to enable a user to query the drug and medical condition library 2105, which is representative of the drug and medical condition library 2015 of FIGS. 20.

The user can select a search field 2110 and can enter a search term 2115 based on the selected search field 2110. In this example scenario, the user has selected the search field 2110 to be a field for receiving an NDC, and the user has entered the following NDC: “0002-7510.” In some cases, the embodiments can automatically determine the search field based on the text entered as the search term 2115 such that the user does not need to manually select the search field 2110. For example, entering text having the format “xxxx-xxxx” can cause the embodiments to infer that a NDC is being entered.

The embodiments can then execute a query against the drug and medical condition library 2105 based on the entered search term 2115. A result of this particular query returns the following information: the drug name 2120, the medical condition 2125 associated with this drug (which was previously parsed based on the acquired text descriptions or which was inferred based on the text descriptions), the NDC 2130, and the description 2135. Of course, other information can be displayed as well, without limit. From the information displayed in the user interface 2100, the user can quickly discern what the drug is and what medical condition is associated with that drug.

### Example Methods

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

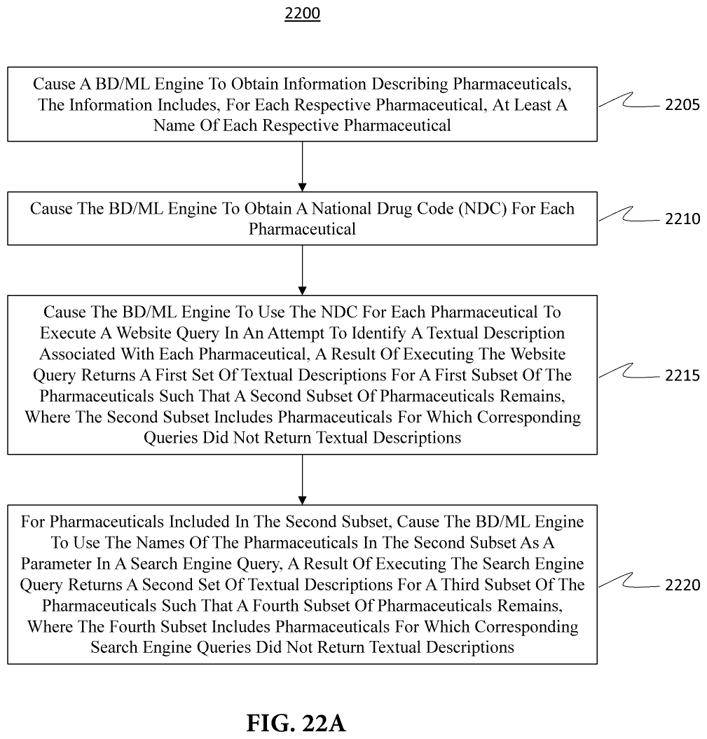

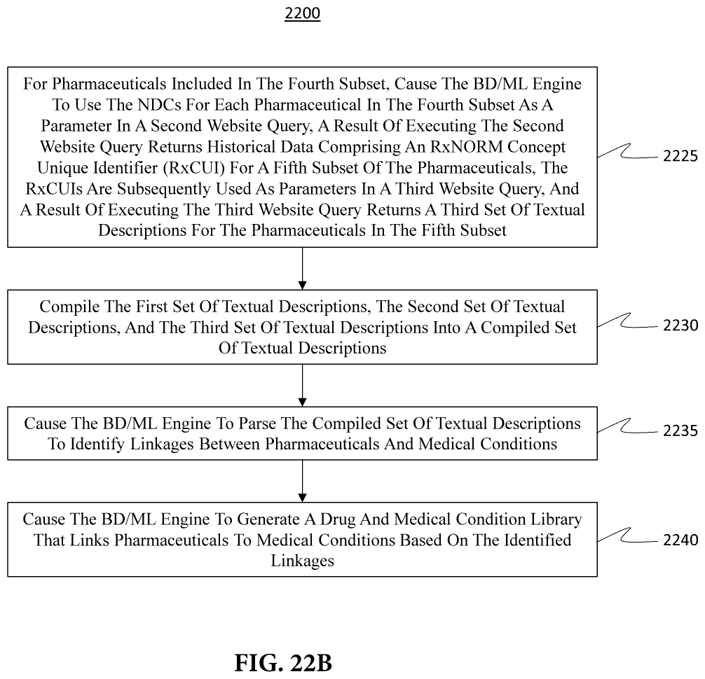

Attention will now be directed to FIGS. 22A and 22B, which illustrate flowcharts of an example method 2200 for performing big data mining to generate a pharmaceutical and medical condition library (e.g., the drug and medical condition library 2105 from FIG. 21). The method 2200 can be implemented by the analysis service 1905 of FIGS. 19 and 20, and in particular can be implemented by the BD/ML engine 1910 of FIG. 19 and/or the NLP engine 2010 of FIG. 20.

Method 2200 includes an act (act 2205) of causing a big data and machine learning (BD/ML) engine (e.g., BD/ML engine 1910 of FIG. 19) to obtain information (e.g., drug information 1915) describing a plurality of pharmaceuticals/drugs. The information includes, for each respective pharmaceutical, at least a name (e.g., drug name 1915A) of each respective pharmaceutical.

Act 2210 includes causing the BD/ML engine to obtain a national drug code (NDC) (e.g., NDC 1915B) for each pharmaceutical.

Act 2215 includes causing the BD/ML engine to use the NDC for each pharmaceutical to execute a website query (e.g., NDC lookup 1920A) in an attempt to identify a textual description (e.g., text descriptions 1920B) associated with each pharmaceutical. A result of executing the website query returns a first set of textual descriptions (e.g., text descriptions 1920B) for a first subset (first subset 1920C) of the pharmaceuticals. Consequently, a second subset (e.g., second subset 1925A) of pharmaceuticals remains, where the second subset includes pharmaceuticals for which the executed queries did not return textual descriptions.

For pharmaceuticals included in the second subset, act 2220 includes causing the BD/ML engine to use the names of the pharmaceuticals in the second subset as a parameter in a search engine query (e.g., search engine lookup 1925B). A result of executing the search engine query returns a second set of textual descriptions (text description 1925C) for a third subset (third subset 1925D) of the pharmaceuticals. Consequently, a fourth subset (fourth subset 1930A) of pharmaceuticals remains, where the fourth subset includes pharmaceuticals for which the executed search engine queries did not return textual descriptions.

Method 2200 continues in FIG. 22B. For pharmaceuticals included in the fourth subset, act 2225 includes causing the BD/ML engine to use the NDCs for each pharmaceutical in the fourth subset as parameters in second website queries (e.g., NDC to RxCUI lookup 1930B). A result of executing the second website queries returns historical data (e.g., historical data 1930C) comprising an RxNorm Concept Unique Identifier (RxCUI) for a fifth subset of the pharmaceuticals. The RxCUIs are subsequently used as parameters in third website queries. A result of executing the third website queries returns a third set of textual descriptions (e.g., text descriptions 1930D) for the pharmaceuticals in the fifth subset (e.g., fifth subset 1930E).

Act 2230 includes compiling the first set of textual descriptions, the second set of textual descriptions, and the third set of textual descriptions into a compiled set of textual descriptions (e.g., text descriptions 2005 from FIG. 20).

Act 2235 includes causing the BD/ML engine to parse the compiled set of textual descriptions to identify linkages between pharmaceuticals and medical conditions. Act 2240 includes causing the BD/ML engine to generate a drug and medical condition library (e.g., drug and medical condition library 2015 from FIG. 20) that links pharmaceuticals to medical conditions based on the identified linkages.

Accordingly, the disclosed embodiments are able to generate a drug and medical condition library that links drugs to specific medical conditions. This library can then be used to generate an index that tracks the costs, or at least the cost fluctuations, for using drugs to treat a particular medical condition.

This index can be configured to not only include the service costs for treating the medical condition, but it can also be configured to include the pharmaceutical costs for treating the medical condition. As a result, a fully comprehensive index can be generated to track and monitor costs for any type of medical condition.

Accordingly, some embodiments access a set of claim forms that are related to an identified medical condition. The embodiments identify, from within the set of claim forms, codes that are determined to be procedure codes. A determination is made as to whether one or more pharmaceuticals are associated with each procedure code in the procedure codes. For procedure codes that have associated pharmaceuticals, the embodiments determine a cost for the pharmaceuticals. The embodiments also weight each of the procedure codes based on whether each procedure code is a descriptor (e.g., text, code, or any other identifying information) for the particular medical condition or is a descriptor for an identified co-morbidity of the particular medical condition. The embodiments determine a cost for the weighted procedure codes. The embodiments use the cost for the weighted procedure codes and the cost for the pharmaceuticals to determine a per capita cost for the medical condition. The embodiments then generate an index for the medical condition based on the per capita cost.

### Example Computer/Computer Systems

Attention will now be directed to FIG. 2300 which illustrates an example computer system 2300 that may include and/or be used to perform any of the operations described herein. Computer system 2300 may take various different forms. For example, computer system 2300 may be embodied as a tablet 2300A, a desktop or a laptop 2300B, a wearable device 2300C, a mobile device, or any other standalone device as shown by the ellipsis 2300D. Computer system 2300 may also be a distributed system that includes one or more connected computing components/devices that are in communication with computer system 2300.

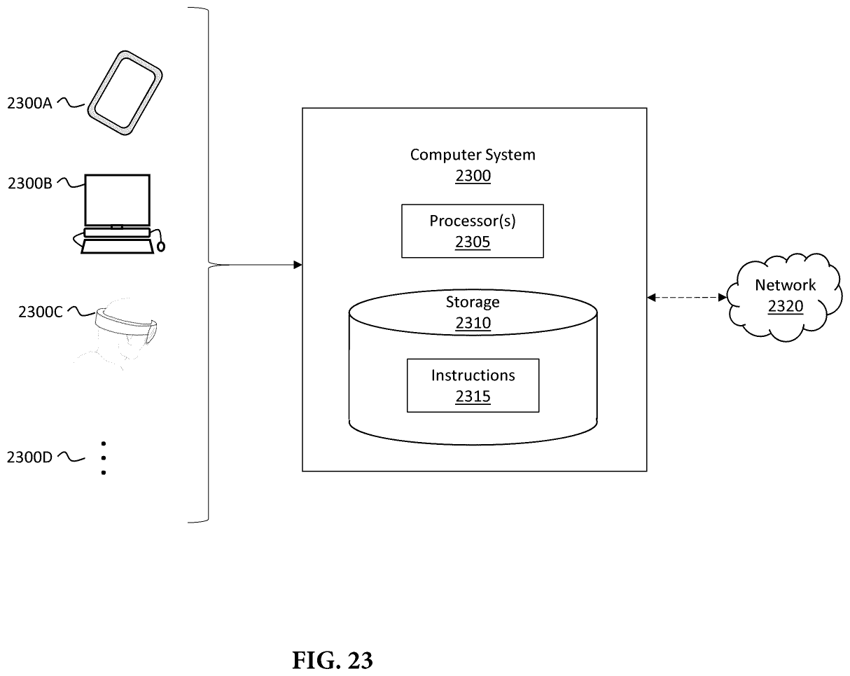

In its most basic configuration, computer system 2300 includes various different components. FIG. 23 shows that computer system 2300 includes one or more processor(s) 2305 (aka a “hardware processing unit”) and storage 2310.

Regarding the processor(s) 2305, it will be appreciated that the functionality described herein can be performed, at least in part, by one or more hardware logic components (e.g., the processor(s) 2305). For example, and without limitation, illustrative types of hardware logic components/processors that can be used include Field-Programmable Gate Arrays (“FPGA”), Program-Specific or Application-Specific Integrated Circuits (“ASIC”), Program-Specific Standard Products (“ASSP”), System-On-A-Chip Systems (“SOC”), Complex Programmable Logic Devices (“CPLD”), Central Processing Units (“CPU”), Graphical Processing Units (“GPU”), or any other type of programmable hardware.

As used herein, the terms “executable module,” “executable component,” “component,” “module,” “engine,” or “service” can refer to hardware processing units or to software objects, routines, or methods that may be executed on computer system 2300. The different components, modules, engines, and services described herein may be implemented as objects or processors that execute on computer system 2300 (e.g. as separate threads).

Storage 2310 may be physical system memory, which may be volatile, non-volatile, or some combination of the two. The term “memory” may also be used herein to refer to non-volatile mass storage such as physical storage media. If computer system 2300 is distributed, the processing, memory, and/or storage capability may be distributed as well.

Storage 2310 is shown as including executable instructions 2315. The executable instructions 2315 represent instructions that are executable by the processor(s) 2305 of computer system 2300 to perform the disclosed operations, such as those described in the various methods.

The disclosed embodiments may comprise or utilize a special-purpose or general-purpose computer including computer hardware, such as, for example, one or more processors (such as processor(s) 2305) and system memory (such as storage 2310), as discussed in greater detail below. Embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are “physical computer storage media” or a “hardware storage device.” Furthermore, computer-readable storage media, which includes physical computer storage media and hardware storage devices, exclude signals, carrier waves, and propagating signals. On the other hand, computer-readable media that carry computer-executable instructions are “transmission media” and include signals, carrier waves, and propagating signals. Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in the form of computer-executable instructions, data, or data structures and that can be accessed by a general-purpose or special-purpose computer.

Computer system 2300 may also be connected (via a wired or wireless connection) to external sensors (e.g., one or more remote cameras) or devices via a network 2320. For example, computer system 2300 can communicate with any number devices or cloud services to obtain or process data. In some cases, network 2320 may itself be a cloud network. Furthermore, computer system 2300 may also be connected through one or more wired or wireless networks to remote/separate computer systems(s) that are configured to perform any of the processing described with regard to computer system 2300.

A “network,” like network 2320, is defined as one or more data links and/or data switches that enable the transport of electronic data between computer systems, modules, and/or other electronic devices. When information is transferred, or provided, over a network (cither hardwired, wireless, or a combination of hardwired and wireless) to a computer, the computer properly views the connection as a transmission medium. Computer system 2300 will include one or more communication channels that are used to communicate with the network 2320. Transmissions media include a network that can be used to carry data or desired program code means in the form of computer-executable instructions or in the form of data structures. Further, these computer-executable instructions can be accessed by a general-purpose or special-purpose computer. Combinations of the above should also be included within the scope of computer-readable media.

Upon reaching various computer system components, program code means in the form of computer-executable instructions or data structures can be transferred automatically from transmission media to computer storage media (or vice versa). For example, computer-executable instructions or data structures received over a network or data link can be buffered in RAM within a network interface module (e.g., a network interface card or “NIC”) and then eventually transferred to computer system RAM and/or to less volatile computer storage media at a computer system. Thus, it should be understood that computer storage media can be included in computer system components that also (or even primarily) utilize transmission media.

Computer-executable (or computer-interpretable) instructions comprise, for example, instructions that cause a general-purpose computer, special-purpose computer, or special-purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the embodiments may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The embodiments may also be practiced in distributed system environments where local and remote computer systems that are linked (either by hardwired data links, wireless data links, or by a combination of hardwired and wireless data links) through a network each perform tasks (e.g. cloud computing, cloud services and the like). In a distributed system environment, program modules may be located in both local and remote memory storage devices.

The present invention may be embodied in other specific forms without departing from its characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

### Incorporation by Specific Reference

The entirety of the following are incorporated herein be specific reference: U.S. Pat. No. 8,271,370 B2, granted Sep. 18, 2012; U.S. Pat. No. 8,370,238 B2, granted Feb. 5, 2003; U.S. Pat. No. 8,510,199 B1, granted Aug. 13, 2013; U.S. Pat. No. 8,374,951 B2, granted Feb. 12, 2001; U.S. Pat. No. 8,370,238 B2, granted Feb. 5, 2013; U.S. Pat. No. 8,306,892 B1, granted Nov. 6, 2012; Publication No. US 2006/0100949 A1, published May 11, 2006; Publication No. US 2012/0215717A1 published Aug. 23, 2013; Publication No. CN111738504A, published Oct. 2, 2020.

1.-6. (canceled)

7. A method for generating an index that is statistically sensitive to a cost of treating a medical condition, where said treating includes both a procedural cost and a pharmaceutical cost, said method comprising:
accessing a set of claim forms that are related to an identified medical condition;
identifying, from within the set of claim forms, codes that are determined to be procedure codes;
determining whether one or more pharmaceuticals are associated with each procedure code in the procedure codes;
for procedure codes that have associated pharmaceuticals, determining a cost for the pharmaceuticals;
weighting each of the procedure codes based on whether each procedure code is a descriptor for the particular medical condition or is a descriptor for an identified co-morbidity of the particular medical condition;
determining a cost for the weighted procedure codes;
using the cost for the weighted procedure codes and the cost for the pharmaceuticals to determine a per capita cost for the medical condition; and
generating an index for the medical condition based on the per capita cost,
wherein the weighting is performed by applying an impact factor to the procedure codes, and wherein the impact factor is defined as a number of times that the procedure code is identified as being associated with a diagnostic code for the identified medical condition within the set of claim forms divided by a total number of times that the procedure code is identified within the set of claim forms.

8.-12. (canceled)

13. A method for generating an index that is statistically sensitive to a cost of treating a medical condition, where said treating includes both a procedural cost and a pharmaceutical cost, said method comprising:
accessing a set of claim forms that are related to an identified medical condition;
identifying, from within the set of claim forms, codes that are determined to be procedure codes;
determining whether one or more pharmaceuticals are associated with each procedure code in the procedure codes;
for procedure codes that have associated pharmaceuticals, determining a cost for the pharmaceuticals;
weighting each of the procedure codes based on whether each procedure code is a descriptor for the particular medical condition or is a descriptor for an identified co-morbidity of the particular medical condition;
determining a cost for the weighted procedure codes;
using the cost for the weighted procedure codes and the cost for the pharmaceuticals to determine a per capita cost for the medical condition; and
generating an index for the medical condition based on the per capita cost,
wherein the method further includes using a big data and machine learning (BD/ML) engine to obtain information describing multiple pharmaceuticals,
wherein the BD/ML engine obtains a national drug code (NDC) for each pharmaceutical in said multiple pharmaceuticals,
wherein the BD/ML engine is caused to use the NDC for each pharmaceutical to execute a website query in an attempt to identify a textual description associated with each pharmaceutical,
wherein a result of executing the website query returns a first set of textual descriptions for a first subset of the pharmaceuticals, and
wherein a second subset of pharmaceuticals remains, where the second subset includes pharmaceuticals for queries that did not return textual descriptions.

14. The method of claim 13, wherein, for pharmaceuticals included in the second subset, the BD/ML engine is caused to use names of pharmaceuticals in the second subset as a parameter in a search engine query.

15. The method of claim 14, wherein a result of executing the search engine query returns a second set of textual descriptions for a third subset of pharmaceuticals.

16. The method of claim 15, wherein a fourth subset of pharmaceuticals remains, where the fourth subset includes pharmaceuticals for search engine queries that did not return textual descriptions, wherein, for pharmaceuticals included in the fourth subset, the BD/ML engine is caused to use the NDCs for each pharmaceutical in the fourth subset as a parameter in a second website query, and wherein a result of executing the second website query returns historical data comprising an RxNorm Concept Unique Identifier (RxCUI) for a fifth subset of the pharmaceuticals.

17. A method for generating an index that is statistically sensitive to a cost of treating a medical condition, said method comprising:
accessing a plurality of claim forms for patients who are identified as having a particular medical condition, wherein the plurality of claim forms include claim forms related to the particular medical condition and claim forms that are not related to the particular medical condition;
filtering the plurality of claim forms to remove claim forms that are not related to the particular medical condition, wherein said filtering is performed by:
performing image segmentation to identify a plurality of claim codes from among the plurality of claim forms;
for each identified claim code in the plurality of claim codes, performing a syntactical analysis on a corresponding description for said each identified claim code to determine whether said each identified claim code is related to the particular medical condition; and
based on the syntactical analysis, removing claim forms that do not have at least one claim code related to the particular medical condition while preserving claim forms that do have at least one claim code related to the particular medical condition even if said claim forms that do have at least one claim code related to the particular medical condition have other claim codes that are not related to the particular medical condition, such that a set of claim forms remain and such that the set of claim forms include claim codes that are related to the particular medical condition and claim codes that are suspected of being co-morbidities to the particular medical condition;
identifying, from within the set of claim forms, codes that are determined to be procedure codes;
weighting each procedure code in said procedure codes based on whether the procedure code is related to the particular medical condition or is related to a co-morbidity of the particular medical condition, wherein said weighting is performed by applying an impact factor to the procedure codes, and wherein the impact factor is defined as a number of times that said procedure code is identified as being associated with a diagnostic code for the particular medical condition within the set of claim forms divided by a total number of times that the procedure code is identified within the set of claim forms;
determining a cost for each weighted procedure code;
using the cost for each weighted procedure code to determine a per capita cost; and
generating an index based on the per capita cost.

18. The method of claim 17, further comprising:
causing a big data and machine learning (BD/ML) engine to obtain information describing a plurality of pharmaceuticals, wherein the information includes, for each respective pharmaceutical in the plurality, at least a name of said each respective pharmaceutical;
causing the BD/ML engine to obtain a national drug code (NDC) for each pharmaceutical in the plurality;
causing the BD/ML engine to use the NDCs for the pharmaceuticals in the plurality to execute website queries in an attempt to identify textual descriptions for the pharmaceuticals, wherein results of executing the website queries return a first set of textual descriptions for a first subset of the pharmaceuticals in the plurality such that a second subset of pharmaceuticals remains, where the second subset includes pharmaceuticals for which the website queries did not return textual descriptions;
for pharmaceuticals included in the second subset, causing the BD/ML engine to use the names of the pharmaceuticals in the second subset as parameters in search engine queries, wherein results of executing the search engine queries return a second set of textual descriptions for a third subset of the pharmaceuticals in the plurality such that a fourth subset of pharmaceuticals remains, where the fourth subset includes pharmaceuticals for which the search engine queries did not return textual descriptions;
for pharmaceuticals included in the fourth subset, causing the BD/ML engine to use the NDCs for the pharmaceuticals in the fourth subset as parameters in second website queries, wherein results of executing the second website queries return historical data comprising RxNorm Concept Unique Identifiers (RxCUIs) for a fifth subset of the pharmaceuticals in the plurality, wherein the RxCUIs are subsequently used as parameters in third website queries, and wherein results of executing the third website queries return a third set of textual descriptions for the pharmaceuticals in the fifth subset;
compiling the first set of textual descriptions, the second set of textual descriptions, and the third set of textual descriptions into a compiled set of textual descriptions;
causing the BD/ML engine to parse the compiled set of textual descriptions to identify linkages between pharmaceuticals and medical conditions; and
causing the BD/ML engine to generate a drug and medical condition library that links pharmaceuticals to medical conditions based on the identified linkages.

19. A method for generating an index that is statistically sensitive to a cost of treating a medical condition, said method comprising:
accessing a first plurality of patient medical claims, wherein each of the first plurality of patient medical claims comprises one or more procedure code(s) and a corresponding one or more diagnostic code(s) associated with a respective one or more procedure code(s), wherein each of the one or more procedure code(s) relates to a respective medical procedure, service, or supply and is associated with at least one of:
a diagnostic code that is related to a particular medical condition; and
a diagnostic code that is not related to the particular medical condition;
filtering the first plurality of patient medical claims to (i) remove claims that do not include at least one of the one or more procedure code(s) that is associated with a diagnostic code that is related to the particular medical condition and (ii) retain claims that include at least one of the one or more procedure code(s) that is associated with a diagnostic code that is related to the particular medical condition, thereby generating a first subset of patient medical claims that are related to the particular medical condition;
weighting each of the one or more procedure code(s) of the first subset of patient medical claims to determine whether each of the one or more procedure code(s) of the first subset of patient medical claims is related to the particular medical condition or is related to a co-morbidity of the particular medical condition, wherein said weighting is performed by applying an impact factor to each of the one or more procedure code(s) of the first subset of patient medical claims, and wherein the impact factor is defined as a number of times that said each of the one or more procedure code(s) of the first subset of patient medical claims is identified as being associated with at least one diagnostic code that is related to the particular medical condition divided by a total number of times that said each of the one or more procedure code(s) is identified within the first subset of patient medical claims, thereby producing a first set of weighted procedure codes;
accessing a second plurality of patient medical claims, wherein each of the second plurality of patient medical claims comprises one or more undesignated procedure code(s) that do not have a corresponding diagnostic code;
optionally filtering the second plurality of patient medical claims to (i) remove claims that do not include at least one of the one or more procedure code(s) from the first subset of patient medical claims and (ii) retain claims that include at least one of the one or more procedure code(s) from the first subset of patient medical claims, thereby generating a second subset of patient medical claims that are related to the particular medical condition;
weighting each of the one or more procedure code(s) of the second subset of patient medical claims to determine whether each of the one or more procedure code(s) of the second subset of patient medical claims is related to the particular medical condition or is related to a co-morbidity of the particular medical condition, wherein said weighting is performed by applying the impact factor to each of the one or more procedure code(s) of the second subset of patient medical claims, thereby producing a second set of weighted procedure codes;
determining a cost for each medical procedure, service, or supply related to each of said one or more procedure code(s) in the first set of weighted procedure codes and the second set of weighted procedure codes based on a cost of said each of the one or more procedure code(s) reflected in the first plurality of patient medical claims and the second plurality of patient medical claims;
using the cost for each medical procedure, service, or supply to determine a per capita cost for the particular medical condition; and
generating a financial index based on or representing the per capita cost for the particular medical condition.

20. The method of claim 19, further comprising:
causing a big data and machine learning (BD/ML) engine to obtain information describing a plurality of pharmaceuticals, wherein the information includes, for each respective pharmaceutical in the plurality, at least a name of said each respective pharmaceutical;
causing the BD/ML engine to obtain a national drug code (NDC) for each pharmaceutical in the plurality;
causing the BD/ML engine to use the NDCs for the pharmaceuticals in the plurality to execute website queries in an attempt to identify textual descriptions for the pharmaceuticals, wherein results of executing the website queries return a first set of textual descriptions for a first subset of the pharmaceuticals in the plurality such that a second subset of pharmaceuticals remains, where the second subset includes pharmaceuticals for which the website queries did not return textual descriptions;
for pharmaceuticals included in the second subset, causing the BD/ML engine to use the names of the pharmaceuticals in the second subset as parameters in search engine queries, wherein results of executing the search engine queries return a second set of textual descriptions for a third subset of the pharmaceuticals in the plurality such that a fourth subset of pharmaceuticals remains, where the fourth subset includes pharmaceuticals for which the search engine queries did not return textual descriptions;
for pharmaceuticals included in the fourth subset, causing the BD/ML engine to use the NDCs for the pharmaceuticals in the fourth subset as parameters in second website queries, wherein results of executing the second website queries return historical data comprising RxNorm Concept Unique Identifiers (RxCUIs) for a fifth subset of the pharmaceuticals in the plurality, wherein the RxCUIs are subsequently used as parameters in third website queries, and wherein results of executing the third website queries return a third set of textual descriptions for the pharmaceuticals in the fifth subset;
compiling the first set of textual descriptions, the second set of textual descriptions, and the third set of textual descriptions into a compiled set of textual descriptions;
causing the BD/ML engine to parse the compiled set of textual descriptions to identify linkages between pharmaceuticals and medical conditions; and
causing the BD/ML engine to generate a drug and medical condition library that links pharmaceuticals to medical conditions based on the identified linkages.
